(19) United States
(12) Patent Application Publication
(10) Pub. No.: US 2024/0081916 A1
(43) Pub. Date: Mar. 14, 2024

(54) COMPUTER ASSISTED SURGICAL SYSTEMS AND METHODS

(71) Applicants: Brent FELIX, Salt Lake City, UT (US); Corey WRIDE, Sandy, UT (US)

(72) Inventors: Brent FELIX, Salt Lake City, UT (US); Corey WRIDE, Sandy, UT (US)

(21) Appl. No.: 18/244,826

(22) Filed: Sep. 11, 2023

Related U.S. Application Data
(60) Provisional application No. 63/405,450, filed on Sep. 11, 2022.

(51) Int. Cl. A61B 34/20; A61B 34/00; A61B 90/00

(52) U.S. Cl. CPC A61B 34/20; A61B 34/25; A61B 90/37; A61B 2034/2057; A61B 2034/2059; A61B 2034/256; A61B 2034/258; A61B 2090/365; A61B 2090/376

(57) ABSTRACT

A computer assisted medical procedure system includes at least one registering computing device, a medical device locator operatively coupled to a medical device placed within a medical procedure theater, wherein the at least one registering computing device determines the location of the medical device locator and medical device relative to a patient, and a patient position fiducial associated with anatomy of a patient being subjected to a medical procedure wherein the at least one registering computing device detects the position of the patient position fiducial relative to the medical device locator in order to determine a relative position of the medical device to the anatomy of the patient.

### CROSS-REFERENCE TO RELATED APPLICATIONS

This application claims the benefit of U.S. Provisional Patent Application Ser. No. 63/405,450 entitled “COMPUTER ASSISTED MEDICAL SYSTEMS AND METHODS,” filed on Sep. 11, 2022, the disclosure of which is incorporated herein by reference in its entirety.

### TECHNICAL FIELD

The present disclosure relates to computer assisted medical procedures. More specifically, the present disclosure relates to a system used to provide location references of a patient's body relative to instruments used to perform a medical procedure.

### BACKGROUND

Medical procedures performed on a patient may include a variety of procedures that include injections, spinal surgery, heart surgery, brain surgery, and other procedures that rely on a doctor's ability to accurately locate surgical sites. Accuracy is especially important after the patient has moved or was moved on the operating table. In some medical procedures, the patient may remain conscious (e.g., injections at a spinal cord) with local or no anesthesia being employed. Properly injecting a medication at an injection site, for example, may prove difficult if the patient moves as the doctor is preparing to perform the injection, identifies the injection site, and attempts to inject the patient at that site on the patient's body.

### SUMMARY

The various systems and methods of the present disclosure have been developed in response to the present state of the art, and in particular, in response to the problems and needs in the art that have not yet been fully solved by currently available medical procedure systems. The systems and methods of the present disclosure may provide a medical procedure system that detects an absolute location on a patient's body where a medical procedure is initiated as well as internal anatomy of the patient's body. In an embodiment, this absolute location of the site of the patient's anatomy may be a location on and within a patient's body that the medical procedure is to be conducted. In an embodiment, the location of the site of the patient's body where the medical procedure is conducted on may be identified using a fluoroscopy device.

To achieve the foregoing, and in accordance with the disclosure as embodied and broadly described herein, the present specification describes a computer assisted medical procedure system that includes a calibrating computing device. The system may also include a medical device placed within a medical procedure theater and a medical device locator operatively coupled to the medical device. The calibrating computing device determines the location of the medical device locator relative to a patient. A patient position fiducial associated with a patient being subjected to a medical procedure may be used such that the calibrating computing device detects the position of the patient position fiducial relative to the medical device locator in order to determine a relative position of the medical device to the anatomy of the patient.

These and other features and advantages of the present disclosure will become more fully apparent from the following description and appended claims, or may be learned by the practice of the disclosure as set forth hereinafter.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the specification's scope, the exemplary embodiments of the present specification will be described with additional specificity and detail through use of the accompanying drawings in which:

FIG. 1 is a graphical diagram of a computer assisted medical procedure system according to one embodiment;

FIG. 2 is a graphical diagram of a developer application program interface (API) testing graphical user interfaces (GUIs) provided to a developer and a relational database program GUI provided to the developer of a computer assisted medical procedure system according to one embodiment;

FIG. 3 is a graphical diagram of a computer assisted medical procedure system according to another embodiment;

FIG. 4 is a graphical diagram of a computer assisted medical procedure system during a first step of a calibration process according to one embodiment;

FIG. 5 is a graphical diagram of a computer assisted medical procedure system during a second step of a calibration process according to one embodiment;

FIG. 6 is a graphical diagram of a computer assisted medical procedure system during a third step of a calibration process according to one embodiment;

FIG. 7 is a graphical diagram of a computer assisted medical procedure system during a fourth step of a calibration process according to one embodiment;

FIG. 8 is a graphical diagram of a computer assisted medical procedure system during a fifth step of a calibration process according to one embodiment;

FIG. 9 is a graphical diagram of a computer assisted medical procedure system during a sixth step of a calibration process according to one embodiment;

FIG. 10 is a graphical diagram of a computer assisted medical procedure system during a seventh step of a calibration process according to one embodiment;

FIG. 11 is a graphical diagram of a computer assisted medical procedure system during a first step of a medical procedure according to one embodiment;

FIG. 12 is a graphical diagram of a computer assisted medical procedure system during a second step of a medical procedure according to one embodiment;

FIG. 13 is a graphical diagram of a computer assisted medical procedure system during a third step of a medical procedure according to one embodiment;

FIG. 14 is a graphical diagram of a computer assisted medical procedure system during a fourth step of a medical procedure according to one embodiment;

FIG. 15 is a graphical diagram of a computer assisted medical procedure system during a fifth step of a medical procedure according to one embodiment;

FIG. 16 is a graphical diagram of a computer assisted medical procedure system during a sixth step of a medical procedure according to one embodiment;

FIG. 17 is a graphical diagram of a computer assisted medical procedure system during a seventh step of a medical procedure according to one embodiment;

FIG. 18 is a graphical diagram of a computer assisted medical procedure system during an eighth step of a medical procedure according to one embodiment;

FIG. 19 is a graphical diagram of a GUI associated with the operation of the computer assisted medical procedure system during the eight step of a medical procedure according to one embodiment;

FIG. 20 is a graphical diagram of a computer assisted medical procedure system during a ninth step of a medical procedure according to one embodiment;

FIG. 21 is a graphical diagram of a computer assisted medical procedure system during a tenth step of a medical procedure according to one embodiment;

FIG. 22 is a diagram of a plurality of spatial object location images for a computer assisted medical procedure system according to one embodiment;

FIG. 23 is a diagram of a spatial object location image with hardware associated with a computer assisted medical procedure system according to one embodiment;

FIG. 24 is a diagram of a plurality of medical device locators for a computer assisted medical procedure system according to one embodiment;

FIG. 25 is a diagram of a spatial anchor used within a medical procedure theater according to and embodiment;

FIG. 26 is a diagram of a first image capturing device and a second image capturing device operatively coupled to a support structure according to an embodiment;

FIG. 27 is a diagram of a 3D box target used as a visual target for a stereoscopic camera system accord to an embodiment;

FIG. 28 is a diagram of a 3D box target with edges highlighted by a hardware processing device of a medical server indicating an outline of the 3D box target according to an embodiment;

FIG. 29 is a diagram of a 3D box target with first detected edges and second detected edges highlighted by a hardware processing device of a medical server indicating an outline of the 3D box target according to an embodiment;

FIG. 30 is a block diagram showing a triangulation process of triangulating a 3D box target within a 3D space according to an embodiment;

FIG. 31 is a diagram of a first 3D box target and second 3D box target with first detected edges and second detected edges, respectively, as indicated by a hardware processing device of a medical server indicating an outline of the first 3D box target and second 3D box target according to another embodiment;

FIG. 32 is a diagram of a first 3D box target with derived edges and a second detected edges with derived edges highlighted by a hardware processing device of a medical server according to another embodiment;

FIG. 33 is a diagram of a subordinate device support structure used to hold a subordinate device used to track the location of a medical device in a medical procedure theater according to an embodiment;

FIG. 34 is a diagram of a subordinate device support structure used to hold a subordinate device within a 3D box target used to track the location of a medical device in a medical procedure theater according to an embodiment;

FIG. 35 is a diagram of a pair of primary devices consisting of a first image capturing device and second image capturing device relative to a holding surface 1210 for a subordinate device according to an embodiment;

FIG. 36 is a diagram showing a c-arm shaped fluoroscopy device to be calibrated using a plurality of 3D box targets according to an embodiment;

FIG. 37 is a diagram of a fluoroscopy device within a medical procedure theater during a calibration check using a registering computing device according to an embodiment;

FIG. 38 is a diagram of a partially opaque fiducial tray including a plurality of partially opaque fiducials formed therein for calculating a pixel-to-meter ratio value on a DICOM file according to an embodiment;

FIG. 39 is a diagram depicting a trackable medical needle used in a medical procedure according to an embodiment;

FIG. 40 is a diagram depicting the trackable medical needle with a needle tracking array operatively coupled to a proximal end of the trackable medical needle according to an embodiment.

### DETAILED DESCRIPTION

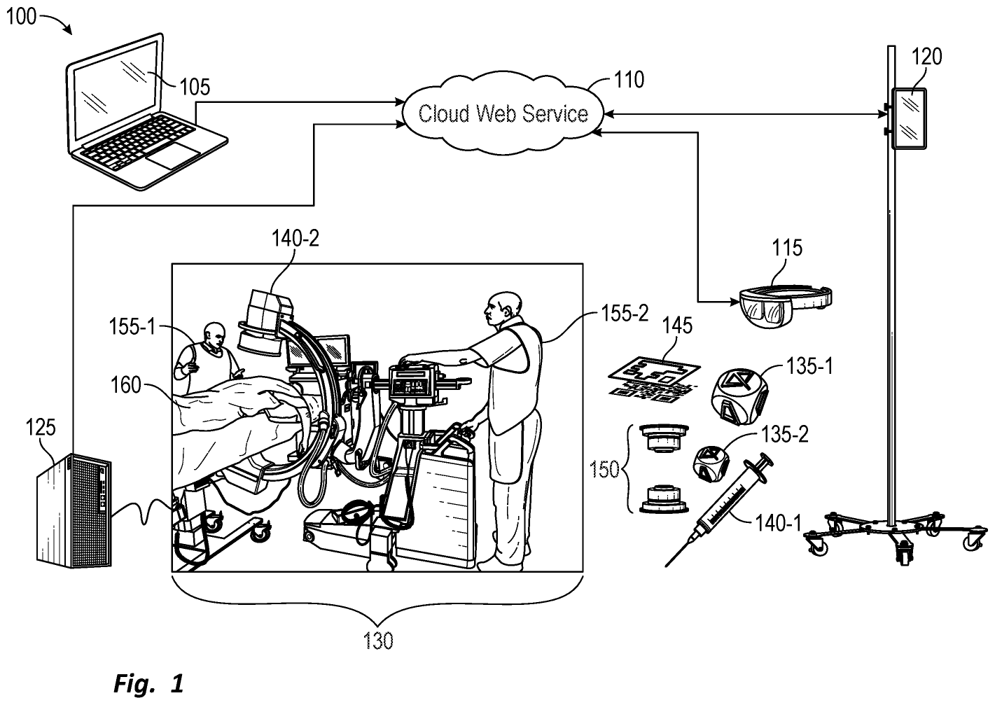

Exemplary embodiments of the disclosure will be best understood by reference to the drawings, wherein like parts are designated by like numerals throughout. It will be readily understood that the components of the disclosure, as generally described and illustrated in the Figures herein, could be arranged and designed in a wide variety of different configurations. Thus, the following more detailed description of the embodiments of the apparatus, system, and method, as represented in FIGS. 1 through 39, is not intended to limit the scope of the disclosure, as claimed, but is merely representative exemplary of exemplary embodiments of the disclosure.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Referring to FIG. 1, a graphical diagram of a computer assisted medical procedure system 100 according to one embodiment of the disclosure is shown. The computer assisted medical procedure system 100 may be used during a medical procedure conducted on a user. In the context of the present specification, the medical procedure discussed may include an injection into or around the patient's 160 spinal cord or backbone herein referred to as the “medical procedure.” However, it is appreciated that other medical procedures may be conducted by the medical personnel 155-1, 155-2 within a medical procedure theater 130 and the present specification contemplates the use of the computer assisted medical procedure system 100 and its associated methods for those medical procedures.

The computer assisted medical procedure system 100 includes an administrator computing device 105. The administrator computing device 105 includes any instrumentality or aggregate of instrumentalities operable to compute, classify, process, transmit, receive, retrieve, originate, switch, store, display, manifest, detect, record, reproduce, handle, or use any form of information, intelligence, or data for business, scientific, control, entertainment, or other purposes. For example, the administrator computing device 105 can be a personal computer, mobile device (e.g., personal digital assistant (PDA) or smart phone), server (e.g., blade server or rack server), a consumer electronic device, a network server or storage device, a network router, switch, or bridge, wireless router, or other network communication device, a network connected device (cellular telephone, tablet device, etc.), IoT computing device, wearable computing device, a set-top box (STB), a mobile information handling system, a palmtop computer, a laptop computer, a desktop computer, a convertible laptop, a tablet, a smartphone, a communications device, an access point (AP), a base station transceiver, a wireless telephone, a control system, a camera, a scanner, a printer, a personal trusted device, a web appliance, or any other suitable machine capable of executing a set of instructions (sequential or otherwise) that specify actions to be taken by that machine, and can vary in size, shape, performance, price, and functionality. The administrator computing device 105 may allow an administrator to access medical records maintained on a medical server 125, interface with the other devices of the computer assisted medical procedure system 100 and provide graphical user interfaces (GUI) to an administrator, among other tasks. The administrator computing device 105 may provide access to medical records for an administrator or other authorized personal. In an embodiment, the administrator may provide a username and/or password via a GUI on the administrator computing device 105 to gain access to the medical records. Because the medical records are subject to the health insurance portability and accountability act (HIPAA) or other laws and regulations, the securing of these medical records may be accomplished by requiring the username and password at the administrator computing device 105.

The administrator computing device 105 may be operatively coupled to a medical server 125 via a cloud web service 110. The medical server 125 may maintain, among other data, any digital imaging and communication in medicine (DICOM) data including images taken by the second medical device 140-2 (e.g., an x-ray machine). The administrator computing device 105 may be operatively coupled to the medical server 125 via the cloud web services 110 by implementing a wireless or wired connection to the cloud web services 110. The cloud web services 110, in an embodiment, may be part of a wide area network (WAN), a local area network (LAN), wireless local area network (WLAN), a wireless personal area network (WPAN), a wireless wide area network (WWAN), or other network. Wireless communication with the cloud web services 110 by the administrator computing device 105 may include any wireless communication protocol.

The computer assisted medical procedure system 100 further includes a medical procedure theater 130 where the medical procedure is conducted on the patient 160. In the example embodiment, the medical procedure theater 130 includes medical devices used to conduct the medical procedure on the patient 160 such as a first medical device 135-1 (e.g., a needle) and a second medical device 135-2 (e.g., an x-ray machine, other projectional radiography devices, or other medical imaging devices). The first medical device 140-1 and second medical device 140-2 may each be used to evaluate the patient and conduct the medical procedure as described herein.

In an embodiment, the medical procedure theater 130 may include a first medical device locator 135-1 and a second medical device locator 135-2 to track, within the medical procedure theater 130, the first medical device 140-1 and second medical device 140-2, respectively. The first medical device locator 135-1 and second medical device locator 135-2 may each be objects that are couplable to the first medical device 140-1 and second medical device 140-2 and is distinguishable among other objects within the medical procedure theater 130. The first medical device locator 135-1 and second medical device locator 135-2 may be detected by one or more of a calibrating computing device 120 and head-mounted display device 115. The calibrating computing device 120 and head-mounted display device 115 may each have a camera or other object tracking device that detects the location of the first medical device locator 135-1 and second medical device locator 135-2 within the medical procedure theater 130 and relative to the head-mounted display device 115 and calibrating computing device 120. In an embodiment, the location of the head-mounted display device 115 and calibrating computing device 120 within the medical procedure theater 130 may be set to an anchoring location with the medical procedure theater 130 while the measurement of the location of the first medical device locator 135-1 with the first medical device 140-1 and the second medical device locator 135-2 with the second medical device 140-2 is described in a three-dimensional cartesian coordinate system or other coordinate system. The imaging devices of the head-mounted display device 115 and/or calibrating computing device 120 may provide the administrator computing device 105 with these coordinates in order to determine the location of the first medical device 140-1 and second medical device 140-2 relative to the user and, as described herein, one or more patient position fiducials 145.

The computer assisted medical procedure system 100 includes one or more patient position fiducials 145. The patient position fiducials 145 may be affixed to the patient 160 and to a medical bed in order to determine the position of the patient 160 within the medical procedure theater 130 and relative to the medical bed. These patient position fiducials 145 may be detected via the second medical device 140-2 where, in the example embodiments, the second medical device 140-2 is an x-ray machine. In order to be detected by the second medical device 140-2, the patient position fiducials 145 may include x-ray opaque ink or other covering that shows through in an x-ray image produced by the second medical device 140-2. The patient position fiducials 145 may be one or more of a quick response (QR) code or an AprilTag. An AprilTag is a visual fiducial system that, when read, conveys less data than a QR code but allows for similar location targeting. In an embodiment, the patient position fiducials 145 may include a center maker that denotes a center location of the patient position fiducials 145.

The computer assisted medical procedure system 100 also, according to an example embodiment, include one or more device alignment caps 150. In the example embodiment where the second medical device 140-2 is an x-ray machine, the device alignment caps 150 may be operatively coupled to an x-ray emission node and an x-ray detection node on the x-ray machine. These device alignment caps 150 may allow a user to calibrate the x-ray machine prior to use on a patient 160.

During operation, the medical personnel 155-1, 155-2 may use one or both of the head-mounted display device 115 or calibrating computing device 120 to guide the medical personnel 155-1, 155-2, as a user interface as well, to a location on the patient's 160 body where, for example, the first medical device 140-1 (e.g., the needle) is to engage the patient's 160 body. In an embodiment, medical device location data describing a location of each of the first medical device 140-1 and second medical device 140-2 obtained from the head-mounted display device 115 and/or calibrating computing device 120 may be provided to the administrator computing device 105. Concurrently, patient location data describing the location of the patient 160 relative to the patient position fiducials 145 presented in the x-ray images obtained by the second medical device 140-2 may also be provided to the administrator computing device 105. This data may be used to know the real-time location of the first medical device 140-1 and second medical device 140-2 relative to the patient 160 and the patient's anatomy in order to perform the medical procedure accurately. With this data, an overlay image of the first medical device 140-1 may be presented to a medical personnel 155-1, 155-2 via a GUI presenting an x-ray image of the user taken by the second medical device 140-2. A location of the overlay image representing the first medical device 140-1 may be updated in real-time on the x-ray image without having to take multiple x-ray images. The medical personnel 155-1, 155-2 may be presented with this GUI representing the x-ray image and overlay of the first medical device 140-1 via the calibrating computing device 120 among other video display devices so that as the first medical device 140-1 is moved towards the patient 160, the medical personnel 155-1, 155-2 may see the location of the first medical device 140-1 relative to the patient 160.

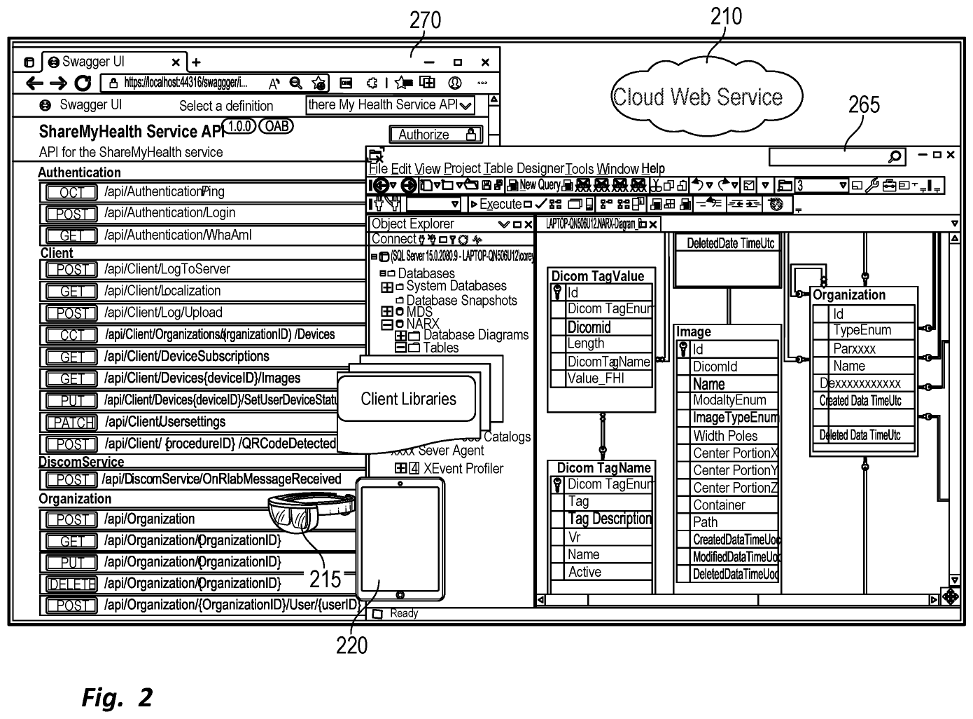

FIG. 2 is a graphical diagram of a developer API testing graphical user interfaces (GUIs) provided to a developer and a relational database program provided to the developer of a computer assisted medical procedure system according to one embodiment of the disclosure. As described herein, the administrator computing device 105 or any other computing device may be provided with a number of GUIs including a relational database program GUI 265 that describes the cloud web services 210 and developer APIs 270 that, when executed by a processor of the computing device, provides for the operation of the system described herein. In one embodiment, the relational database program GUI 265 may provide, in some embodiments, the developer with a relational listing of those resources available to the developer in deploying the systems described herein. The listing of non-volatile resources on the relational database program GUI 265 may include application program interfaces (APIs) that allow the administrator computing device 105 and medical server 125 to interface with the head-mounted display device 215 and calibrating computing device 220 during operation. This allows communication between these devices that include images of a patient's anatomy, medical records associated with the patient, and other medical data. In an embodiment, the relational database program GUI 265 and/or developer API 270 testing GUI may be accessed via an administrator computing device 105 or the medical server 125 among other computing devices associated with the computer assisted medical procedure system described herein in order for a developer to properly deploy the system onto the hardware resources described herein.

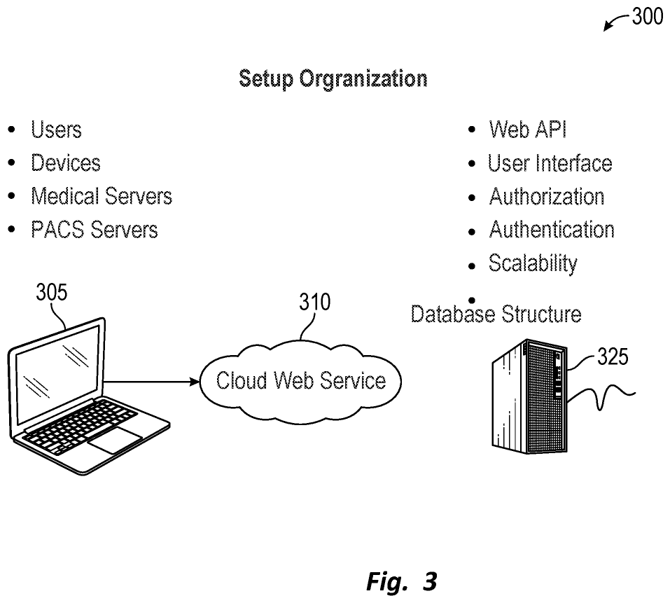

FIG. 3 is a graphical diagram of a computer assisted medical procedure system 300 according to another embodiment of the disclosure. As described in connection with FIG. 1, the computer assisted medical procedure system 300 includes an administrator computing device 105. The administrator computing device 105 includes any instrumentality or aggregate of instrumentalities operable to compute, classify, process, transmit, receive, retrieve, originate, switch, store, display, manifest, detect, record, reproduce, handle, or use any form of information, intelligence, or data for business, scientific, control, entertainment, or other purposes. The administrator computing device 305 may allow an administrator to access medical records maintained on a medical server 325, interface with the other devices of the computer assisted medical procedure system 300 and provide graphical user interfaces (GUI) to an administrator, among other tasks. The administrator computing device 305 may provide access to medical records for an administrator or other authorized personal. In an embodiment, the administrator may provide a username and/or password via a GUI on the administrator computing device 305 to gain access to the medical records. Because the medical records are subject to the health insurance portability and accountability act (HIPAA) or other laws and regulations, the securing of these medical records may be accomplished by requiring the username and password at the administrator computing device 305 among other security features used to secure these confidential records.

The administrator computing device 305 may be operatively coupled to a medical server 325 via a cloud web service 310 in an embodiment. In another embodiment, the medical server 325 may provide any medical files including any DICOM files received from an x-ray machine, for example, to a cloud server for access by the administrator computing device 305. The medical server 325 may maintain, among other data, any digital imaging and communication in medicine (DICOM) data including images taken by the second medical device (e.g., an x-ray machine). The administrator computing device 305 may be provided access to medical records from the medical server 325 via the cloud web services 310 by implementing a wireless or wired connection to the cloud web services 310. In an embodiment, the medical server 325 may be a virtual machine placed on a networked device with data accessible to an administrator computing device 305 as described herein. The cloud web services 310, in an embodiment, may be part of a wide area network (WAN), a local area network (LAN), wireless local area network (WLAN), a wireless personal area network (WPAN), a wireless wide area network (WWAN), or other network. Wireless communication with the cloud web services 310 by the administrator computing device 305 may include any wireless communication protocol.

The general organization of the computer assisted medical procedure system 300 includes mapping of users on the system with their associated devices such as other computing devices used to access the cloud web services 310. The setup of the computer assisted medical procedure system 300 also includes operatively coupling the medical server 325 to the cloud web services 310 and administrator computing device 305 as well as with other computing devices authorized to access the computer assisted medical procedure system 300.

In an embodiment, the setup of the computer assisted medical procedure system 300 may include operatively coupling a picture archiving and communication system (PACS). The PACS may provide services associated with medical imaging technologies and provides economical storage and convenient access to images from multiple different types of medical imaging devices. The PACS server and medical server 325 may, in an embodiment, be a single server that performs the functions of these two servers.

The setup of the computer assisted medical procedure system 300 further includes providing web APIs for a computing device to upload medical files such as DICOM files to a cloud service for access with the medical server 325 or other computing devices within the computer assisted medical procedure system 300. The APIs may provide user interface capabilities at these computing devices that request authorization data and authentication of the user operating the computing devices. Because one or more computing devices may be operatively coupled to the computer assisted medical procedure system 300 the scalability of the data storage and data throughput may be changed based on the number of computing devices.

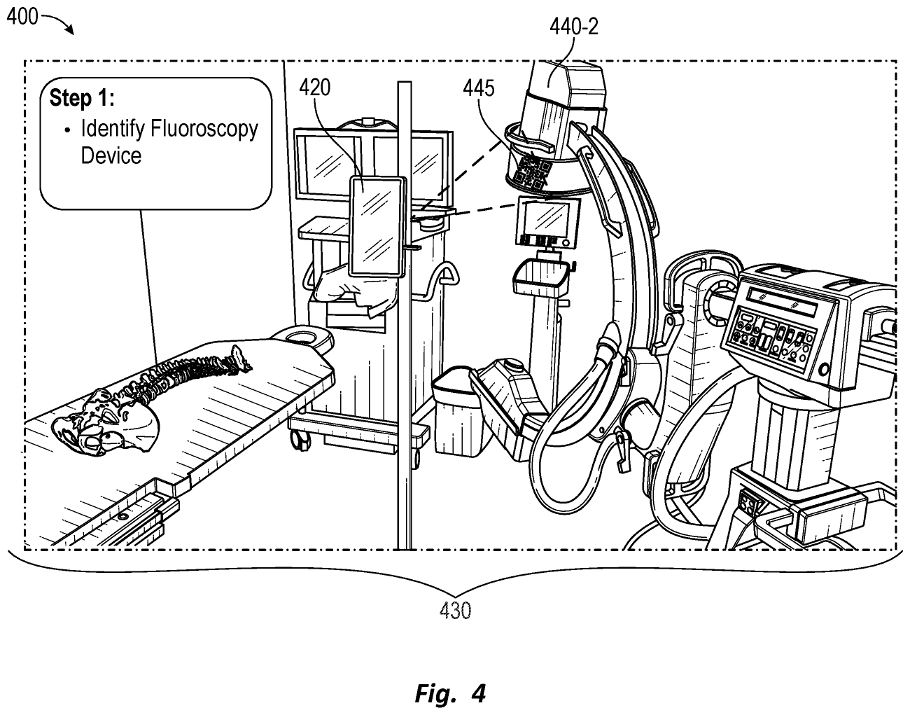

FIGS. 4 through 10 describe a calibration process associated with calibrating a first medical device 440-1 such as an x-ray machine during a surgical procedure such as a medical injection near a spinal cord. In the present specification and in the appended claims, the term “calibration” in the context of FIGS. 4 through 10 is understood as an action or process of correlating readings of an instrument with those of a standard in order to check the accuracy of the associated instruments. In the context of the present specification, this calibration may be conducted on a fluoroscopy device, a camera, a needle, and other devices used during the process described herein. Turning first to FIG. 4, a graphical diagram of a computer assisted medical procedure system 400 during a first step of a calibration process is shown. This first step includes identifying the medical devices associated with the calibration within a medical procedure theater 430. In the example embodiment described herein, the first medical device is a fluoroscopy device 440-2 (e.g., an x-ray machine) used to image bones of a patient including a spine of the patient. The fluoroscopy device 440-2 may be identified using, in an example embodiment, by detecting a fiducial 445 on the fluoroscopy device 440-2 with the calibrating computing device 420. In this embodiment, a camera on the calibrating computing device 420 may be used to scan the fiducial 445 on the fluoroscopy device 440-2. This scan may identify the location of the fluoroscopy device 440-2 within a specific room, identify the specific type of device and qualities of the fluoroscopy device 440-2, among other data associated with the operation of the fluoroscopy device 440-2 within in the computer assisted medical procedure system 400.

In an embodiment, one or more devices may scan the room for this fiducial 445. In an embodiment, the fiducial 445 may alternatively or additionally be located on a wall that is scanned. This scanning may identify the room number, the medical devices located within that room, and may further indicate that the user of the calibrating computing device 420, head-mounted device, or other computing device.

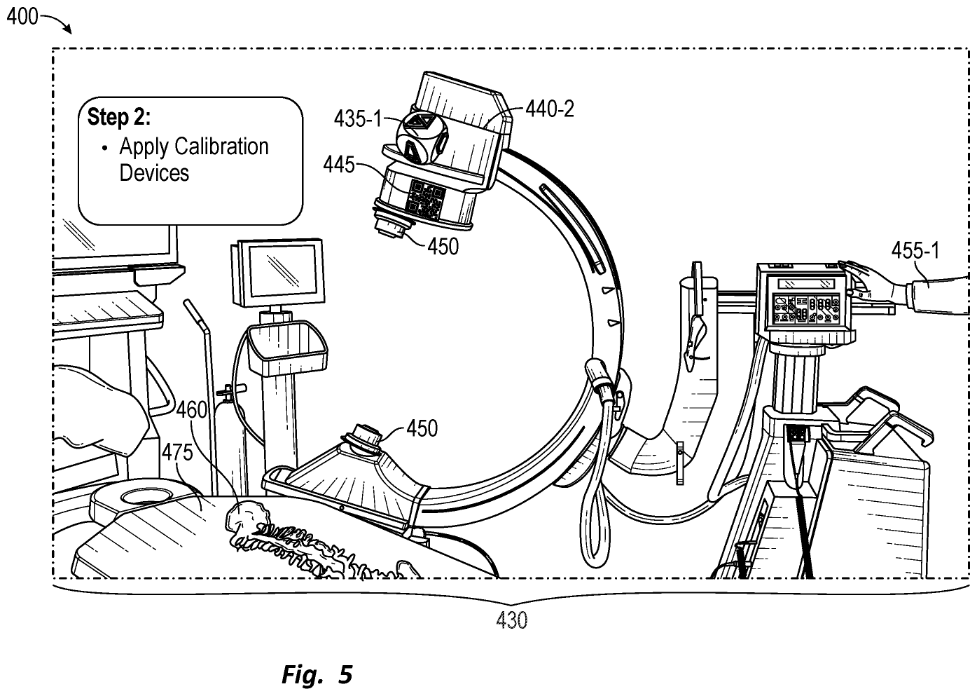

Once identified, this calibration process may continue as shown in FIG. 5. FIG. 5 is a graphical diagram of a computer assisted medical procedure system 400 during a second step of a calibration process according to one embodiment of the disclosure. At this point the device alignment caps 450 may be added to the fluoroscopy device 440-2. In an embodiment, the device alignment caps 450 may be operatively coupled to an x-ray emission node and an x-ray detection node on the fluoroscopy device 440-2. These device alignment caps 450 may allow a user to calibrate the fluoroscopy device 440-2 prior to use on a patient. The device alignment caps 450 may be added to the fluoroscopy device 440-2 by medical personnel 455-1 conducting the calibration of the fluoroscopy device 440-2 in the medical procedure theater 430. A patient surrogate 460 may be placed on a portion of the operation table 475 within the medical procedure theater 430 for preparation during the calibration process.

FIG. 5 also shows that addition of a first medical device locator 435-1 to the fluoroscopy device 440-2. This first medical device locator 435-1 may be in the form of a cube or cube with curved edges. Each side of the first medical device locator 435-1 may include a different surface topology that distinguishes one side from another. By doing this, the calibrating computing device 420, when tracking the location of the fluoroscopy device 440-2, may know the three-dimensional orientation of the fluoroscopy device 440-2 at any time during the calibration process or during operation of the fluoroscopy device 440-2 during a medical procedure.

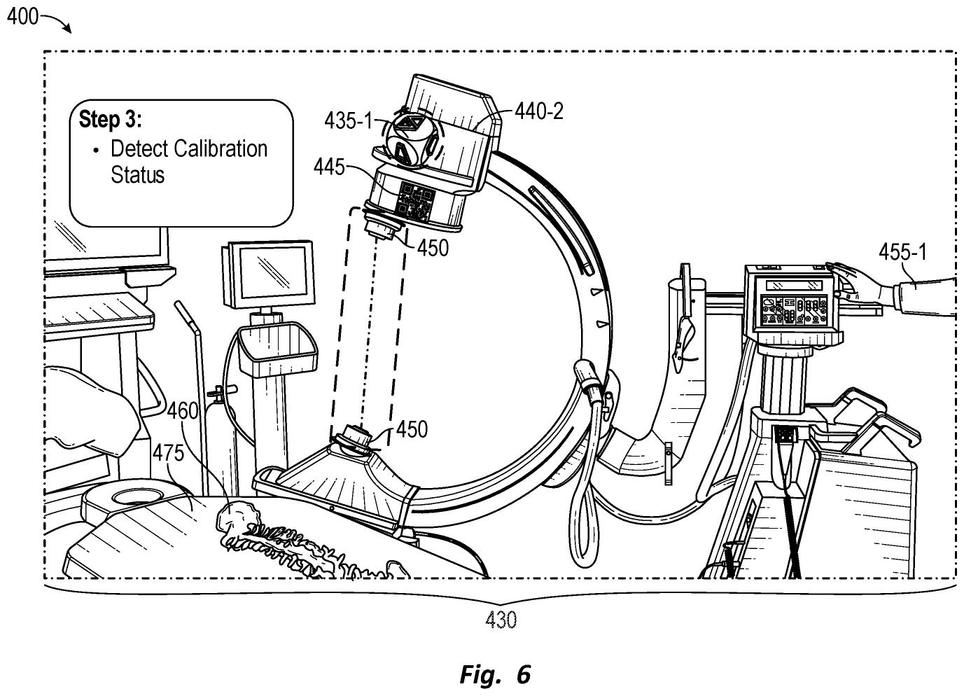

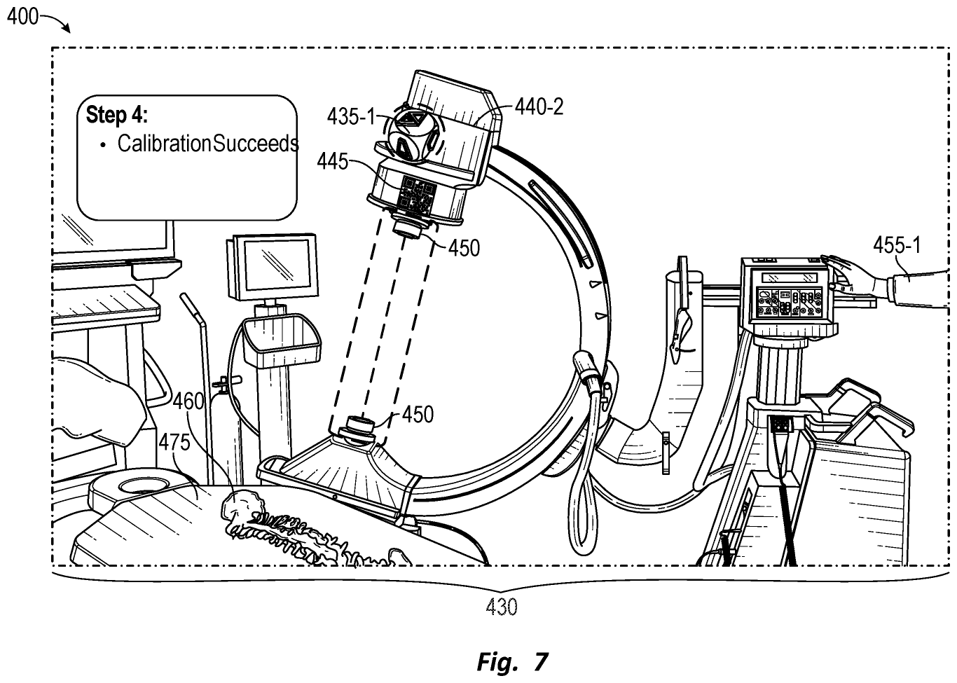

When the device alignment caps 450 have been added to the fluoroscopy device 440-2, the calibration may continue at FIG. 6. FIG. 6 is a graphical diagram of a computer assisted medical procedure system 400 during a third step of a calibration process according to one embodiment of the disclosure. At this step, the fluoroscopy device 440-2 may be turned on to detect the alignment of the x-ray emissions between the x-ray emission node and an x-ray detection node on the fluoroscopy device 440-2. In an embodiment, the location of the emitted x-rays may be determined based on the location of the first medical device locator 435-1.

Where the fluoroscopy device 440-2 has been turned on, the calibration process of the fluoroscopy device 440-2 may continue at FIG. 7. FIG. 7 is a graphical diagram of a computer assisted medical procedure system 400 during a fourth step of a calibration process according to one embodiment of the disclosure. Here the device alignment caps 450 help to align the x-ray emissions and are used, along with the first medical device locator 435-1 to determine the location of the x-ray emissions after the calibration of the fluoroscopy device 440-2 is achieved and alignment of the x-ray emissions is determined.

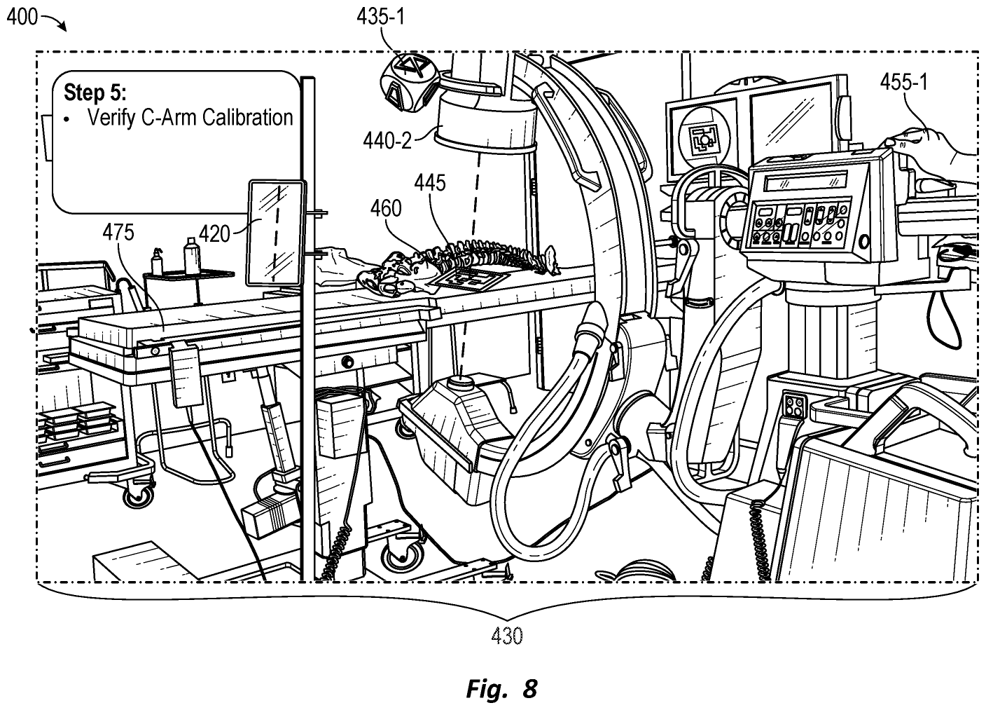

In an embodiment, the calibration of the fluoroscopy device 440-2 may be confirmed as shown in FIG. 8. FIG. 8 is a graphical diagram of a computer assisted medical procedure system 400 during a fifth step of a calibration process according to one embodiment of the disclosure. The calibration of the fluoroscopy device 440-2 relative to a patient surrogate 460 placed on the operation table 475 allows the location of a patient's anatomy to be determined prior to use of the calibrated fluoroscopy device 440-2 in a real medical procedure.

In FIG. 8, a fiducial 445 has been placed on the operation table 475 next to a patient surrogate 460 and the fluoroscopy device 440-2 takes an x-ray image of the area around the patient surrogate 460. As described herein, the fiducial 445 may include x-ray opaque material that allows the resulting x-ray image to show the location of the fiducial 445 relative to the patient surrogate 460. In this embodiment, the fiducial 445 may include a center point that the x-ray emission is directed to in order to center the fiducial 445 within the resulting x-ray image.

As shown in FIG. 8, the resulting x-ray image with a fiducial 445 overlayed on the image. With the first medical device locator 435-1, the device alignment caps 450, and the fiducial 445 next to the patient surrogate 460, the location of the x-ray emission may be determined and presented on a GUI presented to the user on the calibrating computing device 420. The images presented to the user during a medical procedure may also include the x-ray images captured by the fluoroscopy device 440-2 with the fiducial 445 as well.

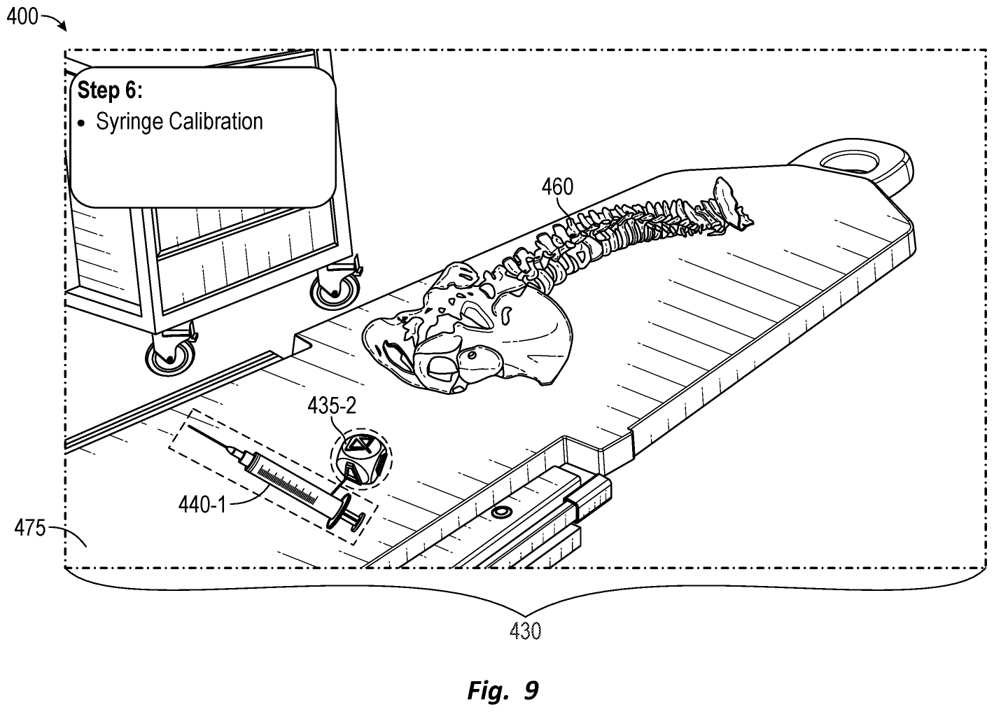

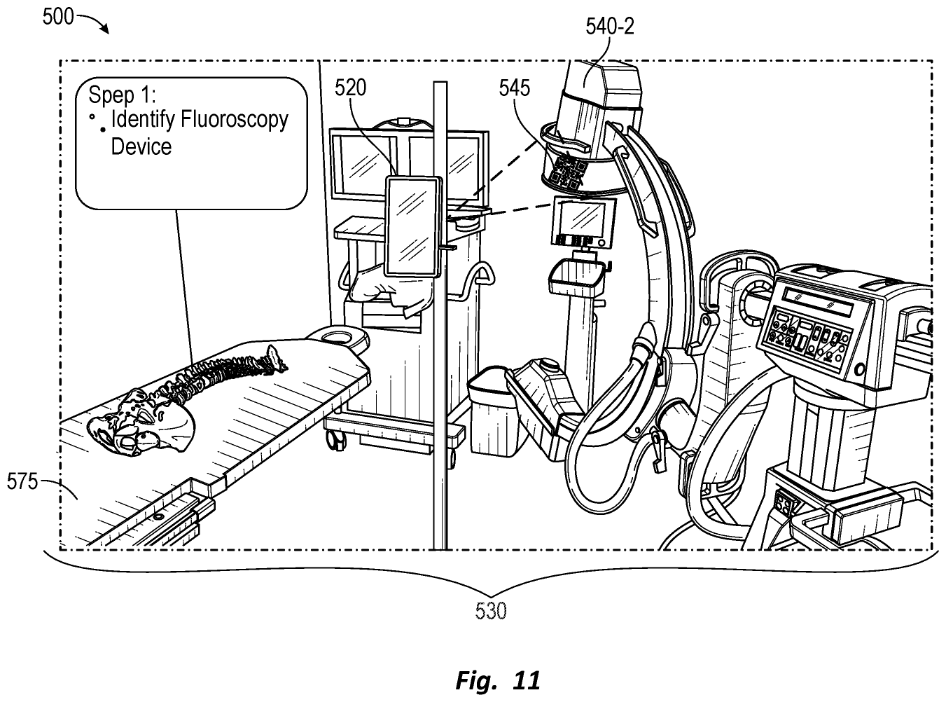

Other medical devices may also be used during the medical procedure and a needle 440-1 as a second medical device is also shown. FIG. 9 is a graphical diagram of a computer assisted medical procedure system 400 during a sixth step of a calibration process according to one embodiment of the disclosure. The needle 440-1, in an embodiment, may be used to provide an injection into, for example, the spine of a patient. The patient surrogate 460 on the operation table 475 shown in FIG. 9 shows a spice and hip bone used in this calibration process and mimicking the medical procedure described in connection with FIGS. 11 through 21.

FIG. 9 shows that a second medical device locator 435-2 is operatively coupled to the needle 440-1 for later location detection of the needle 440-1. As described herein, the second medical device locator 435-2 may also be a cube or cuboid-shaped body that includes distinct faces formed on them. The faces of the sides of the second medical device locator 435-2 may be distinguished by topology, color, or other distinguishing features that allows for a three-dimensional location detection of the second medical device locator 435-2 when installed onto the needle 440-1.

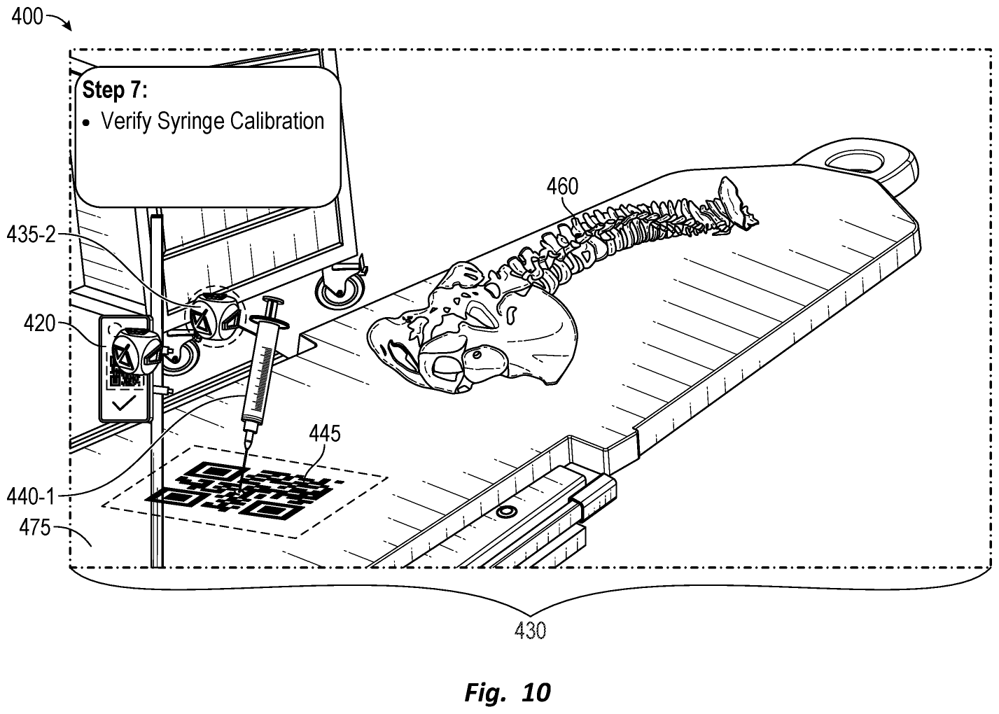

FIG. 10 is a graphical diagram of a computer assisted medical procedure system 400 during a seventh step of a calibration process according to one embodiment of the disclosure. In this process, the needle may be placed adject to or touching a central location of another fiducial 445 placed on the operation table 475. This may be done so that the calibrating computing device 420 may track, in real-time, the location of the needle 440-1 relative to other objects such as the patient surrogate 460 and fiducials 445 located by the patient surrogate 460.

In an embodiment, the fiducial 445 may include a central point where the medical personnel 455-1 may contact the tip of the needle 440-1 during this calibration process. This central point may be provided to the user in order to calibrate the location of the needle 440-1 and second medical device locator 435-2 within the medical procedure theater 430 and relative to the other tracked objects within the medical procedure theater 430.

In an embodiment, the calibrating computing device 420, the medical server, or both may be used to indicate to the medical personnel 455-1 when the tracking of the needle 440-1 and second medical device locator 435-2 is determined. In an embodiment, the processing resources of the calibrating computing device 420, the medical server, or both may be used to complete this calibration process, or any other processing of data described herein. The calibrating computing device 420 may track the needle 440-1 and second medical device locator 435-2 using an imaging device such as a camera on the calibrating computing device 420. In an embodiment, the medical personnel 455-1 may be asked to enter in data descriptive of the type of needle 440-1 being used so that proper calibration may be undertaken. This may allow the calibrating computing device 420 or another computing device to determine the location of the tip of the needle relative to the second medical device locator 435-2 during the medical procedure.

The calibration process described in connection with FIGS. 4 through 10 may be conducted once prior to the execution of any medical procedure in an embodiment. In an embodiment, the calibration of the fluoroscopy device 440-2 and the location determination of the fluoroscopy device 440-2 within the medical procedure theater 430 may be conducted for each medical procedure conducted in the medical procedure theater 430. In an embodiment, the calibration and location determination of the needle 440-1 and its second medical device locator 435-2 may be conducted prior to any medical procedure including those described in connection with FIGS. 11 through 21.

It is appreciated that the calibration process described in connection with FIGS. 4 through 10 may be conducted concurrently in an embodiment. In another embodiment, those processes describing a specific “step” that is being conducted may be conducted in any order without going beyond the scope of the present description.

FIGS. 11 through 21 show an example medical procedure completed after the calibration process described in connection with FIGS. 4 through 10. FIG. 11 is a graphical diagram of a computer assisted medical procedure system 500 during a first step of a medical procedure according to one embodiment of the disclosure. Similar to the calibration process described in FIG. 4, this first step during this medical procedure may include identifying the medical devices associated with the calibration within a medical procedure theater 530. In the example embodiment described herein, the first medical device is a fluoroscopy device 540-2 (e.g., an x-ray machine) used to image bones of a patient including a spine of the patient. The fluoroscopy device 540-2 may be identified using, in an example embodiment, by detecting a fiducial 545 on the fluoroscopy device 540-2 with a registering computing device 520. The registering computing device 520 may be any device that can detect any fiducial device within the medical procedure theater 530. In this embodiment, a camera on the registering computing device 520 may be used to scan the fiducial 545 on the fluoroscopy device 540-2. This scan may identify the location of the fluoroscopy device 540-2 within a specific room, identify the specific type of device and qualities of the fluoroscopy device 540-2, among other data associated with the operation of the fluoroscopy device 540-2 within in the computer assisted medical procedure system 500.

In an embodiment, one or more devices may scan the room for this fiducial 545. In an embodiment, the fiducial 545 may alternatively or additionally be located on a wall that is scanned. This scanning may identify the room number, the medical devices located within that room, and may further indicate that the user of the registering computing device 520, head-mounted device, or other computing device.

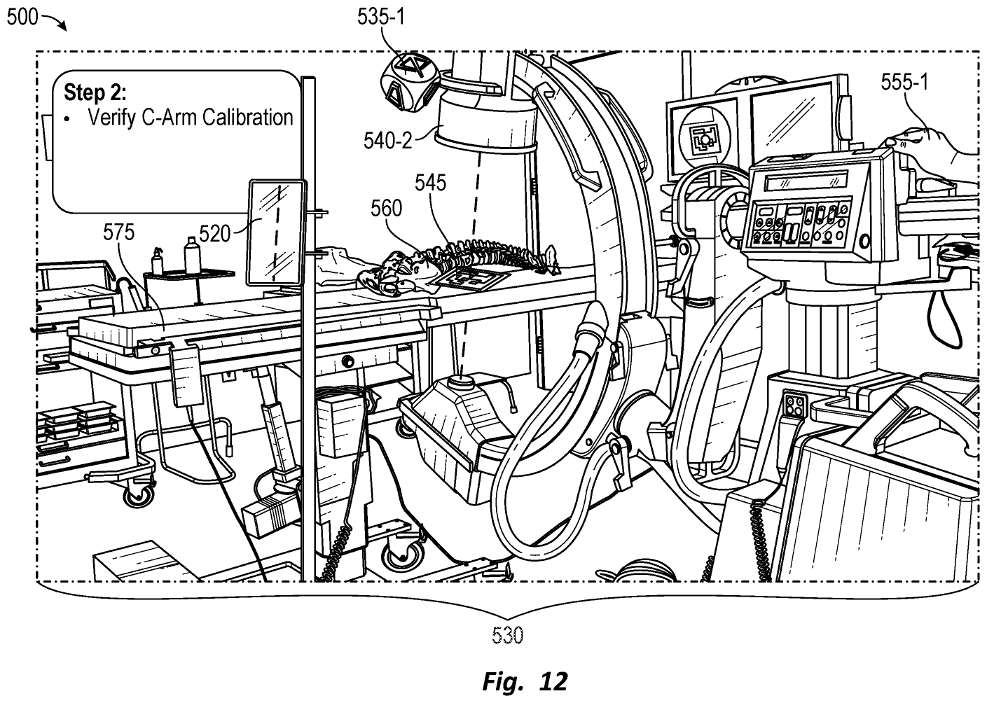

FIG. 12 is a graphical diagram of a computer assisted medical procedure system 500 during a second step of a medical procedure according to one embodiment of the disclosure. During this process, similar to the process associated with FIG. 8, another fiducial 545 has been placed on the operation table 575 next to a patient 560 surrogate and the fluoroscopy device 540-2 takes an x-ray image of the area around the patient 560 surrogate. As described herein, the fiducial 545 may include x-ray opaque material that allows the resulting x-ray image to show the location of the fiducial 545 relative to the patient 560 surrogate. In this embodiment, the additional fiducial 545 may include a center point that the x-ray emission is directed to in order to center the fiducial 545 within the resulting x-ray image.

As shown in FIG. 12, the resulting x-ray image with a fiducial 545 overlayed on the image. With the first medical device locator 535-1, the device alignment caps (not shown), and the additional fiducial 545 next to the patient 560 surrogate, the location of the x-ray emission may be determined and presented on a GUI presented to the user on the registering computing device 520 in an embodiment. The present specification further contemplates that indicating whether the described calibration process has succeeded may be completed via a notification via any presentation to the user including audible indications. FIG. 12 shows that if the AprilTag is centered on DICOM image, then the calibration is verified. The images presented to the user during a medical procedure may also include the x-ray images captured by the fluoroscopy device 540-2 with the fiducial 545 as well.

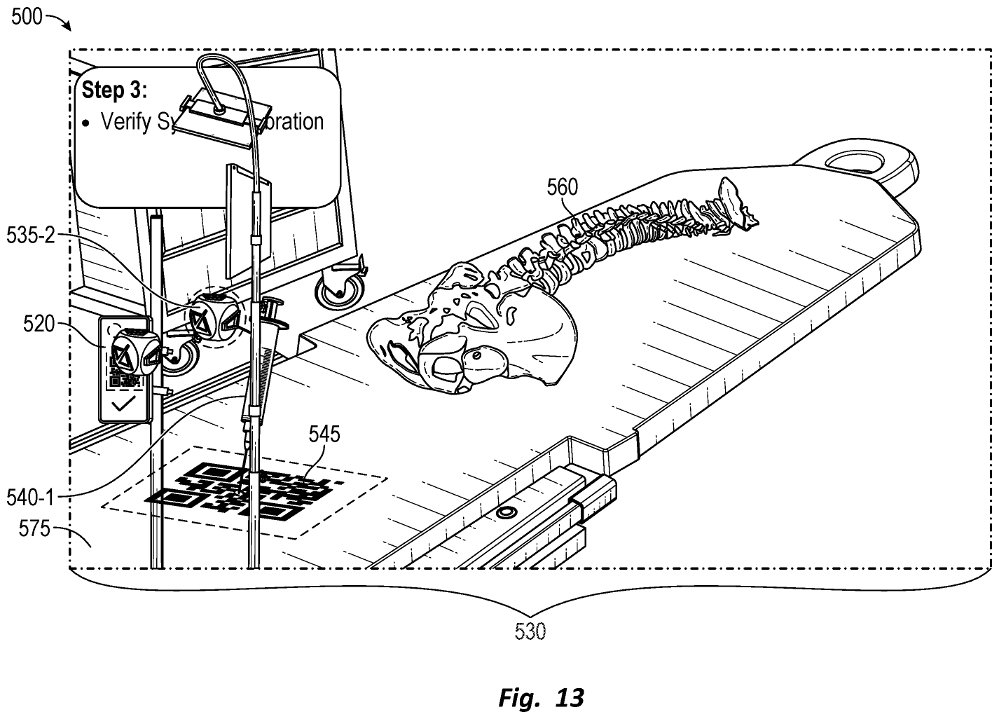

FIG. 13 is a graphical diagram of a computer assisted medical procedure system 500 during a third step of a medical procedure according to one embodiment of the disclosure. In this step, a needle 540-1 may be placed adject to or touching a central location of another fiducial 545 placed on the operation table 575. This may be done so that the registering computing device 520 may track, in real-time, the location of the needle 540-1 relative to other objects such as the patient 560 surrogate and fiducials 545 located by the patient 560 surrogate.

In an embodiment, the fiducial 545 may include a central point where the medical personnel may contact the tip of the needle 540-1 during this calibration process. This central point may be provided to the user in order to calibrate the location of the needle 540-1 and second medical device locator 535-2 within the medical procedure theater 530 and relative to the other tracked objects within the medical procedure theater 530.

In an embodiment, the registering computing device 520 may be used to indicate to the medical personnel when the tracking of the needle 540-1 and second medical device locator 535-2 is determined. The registering computing device 520 may track the needle 540-1 and second medical device locator 535-2 using an imaging device such as a camera on the registering computing device 520. In an embodiment, the medical personnel may be asked to provide data (e.g., via a mouse or keyboard or other input device) descriptive of the type of needle 540-1 being used. This may allow the registering computing device 520 or another computing device to determine the location of the tip of the needle relative to the second medical device locator 535-2 during the medical procedure.

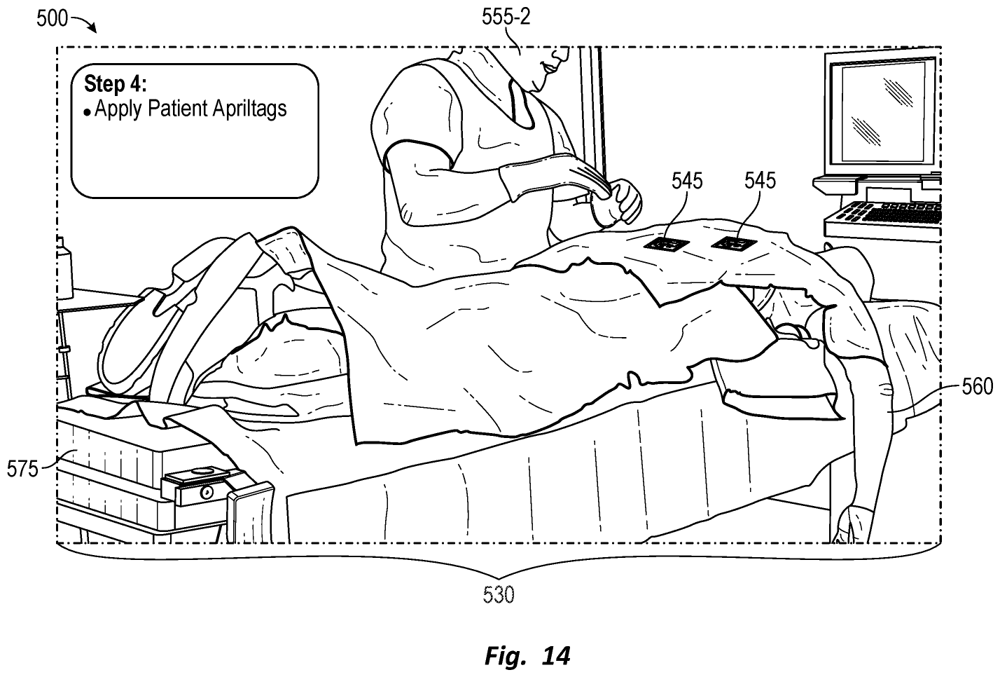

FIG. 14 is a graphical diagram of a computer assisted medical procedure system 500 during a fourth step of a medical procedure according to one embodiment of the disclosure. In this step, a medical personnel 555-1, 555-2 may prep the patient 560 by adding one or more patient fiducials 545 to the body of the patient, on the operation table 575, and/or on a covering over the patient's body, among other locations. Because the fiducials 545 are seen in an x-ray image captured by a fluoroscopy device (not shown in FIG. 14) the patient fiducials 545 may include x-ray opaque materials that allow for the fiducials 545 to be imaged on the resulting x-ray image and seen relative to the anatomy of the patient 560. The medical personnel 555-1, 555-2 may place these patient fiducials 545 near the location of the patient's 560 anatomy where the medical procedure is being performed. In one embodiment, when an x-ray is taken using the fluoroscopy device, the location of the patient relative to the scanline of the fluoroscopy device is known, and it is assumed that the patient does not move. However, to detect potential patient movement, the fiducials 545, which are a special type of target that can be attached to the patient or the equipment nearby, a patient calibration process may be initiated as well. The patient calibration process consists of detecting the distance between at least one fiducial 545 and other fiducials 545 being tracked so that the computer assisted medical procedure system is altered if patient movement has been detected. When patient movement is detected, a medical professional can determine whether to invalidate the x-ray captured by the fluoroscopy device or adjust a medical device's location (e.g., a needle) relative to the patient.

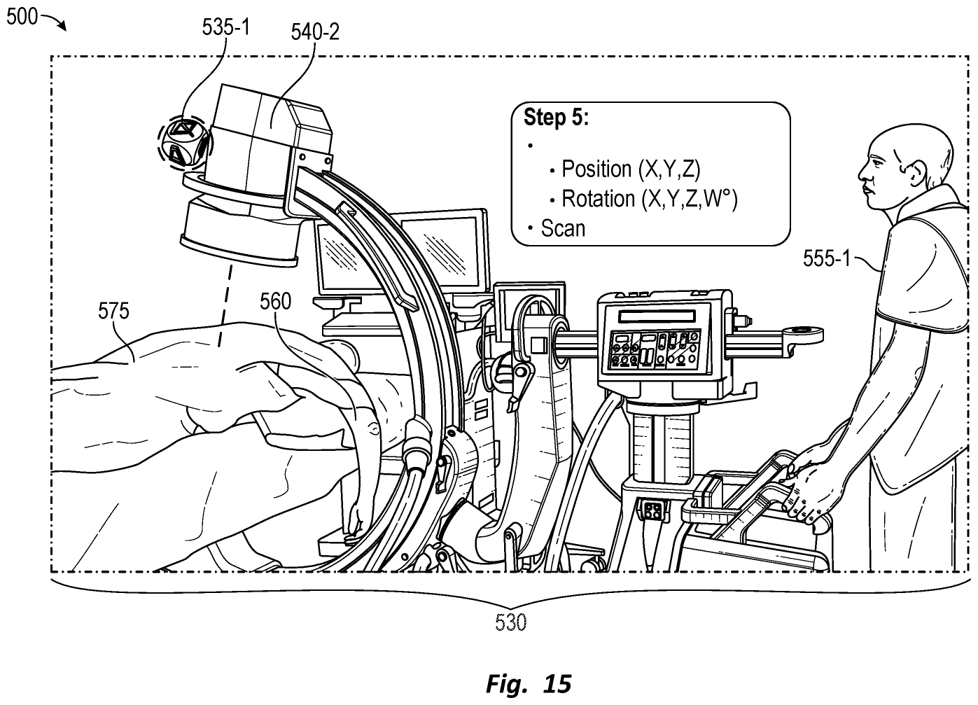

FIG. 15 is a graphical diagram of a computer assisted medical procedure system 500 during a fifth step of a medical procedure according to one embodiment of the disclosure. In this step, the medical personnel 555-1 may move the fluoroscopy device 540-2 in position to capture an x-ray image of the patient's 560 anatomy. As the fluoroscopy device 540-2 is moved into position, the first medical device locator 535-1 coupled thereto may be used to align the fluoroscopy device 540-2 with the proper imaging location. As described herein, the registering computing device 520 or a head-mounted display (not shown) may be used to complete this alignment. The position of the fluoroscopy device 540-2 may include an absolute x, y, and z-position as well as a rotational position of the fluoroscopy device 540-2 in an x. y, z, and w′-position. The x, y, z, dimensions may represent the location of the medical device in 3D space while the w dimension defines the amount of rotation of the medical device.

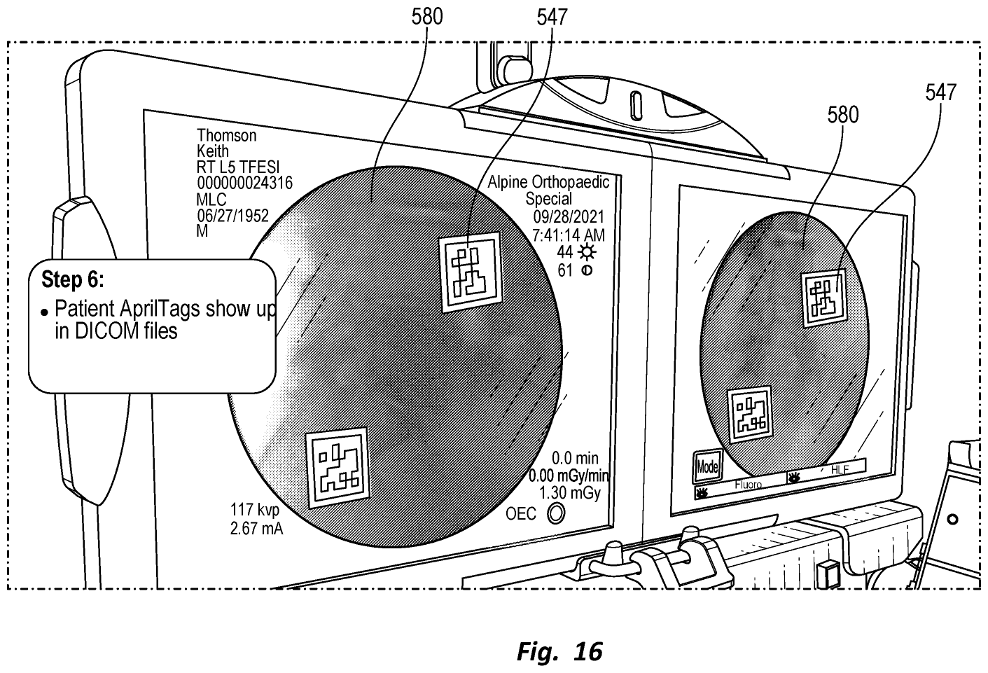

FIG. 16 is a graphical diagram of a computer assisted medical procedure system 500 during a sixth step of a medical procedure according to one embodiment of the disclosure. Here in this step, the resulting x-ray images 580 may be presented to medical personnel 555-1, 555-2 on a display device. This display device may be a dedicated display device, a display device of the registering computing device 520, and/or a display device of a head-mounted display device. As seen in the x-ray images 580, the opaque or partially opaque fiducials 547 have shown up on the x-ray images 580 next to the patient's anatomy that are to be subjected to the medical procedure. As described herein, these x-ray images 580 may be augmented with an overly of other medica devices such as an image of the needle 540-1 as the medical personnel 555-1, 555-2 move the needle 540-1 towards the patient's body. In an embodiment, the scanning of the AprilTag or other location identification tags herein allows for the determination of the pixel-to-meter ratio. A determination of the pixel-to-meter ratio allows for the determination of how big to display the DICOM image on a video display device and further allows for the user to identify which of the x-ray devices are being used to capture the images of the patient's anatomy.

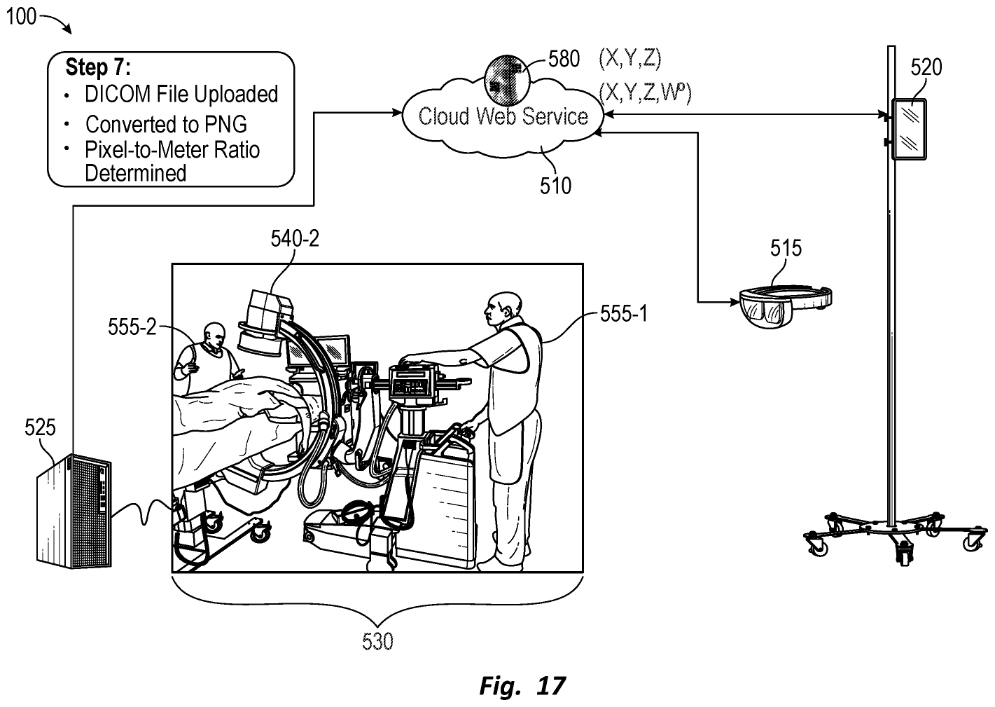

FIG. 17 is a graphical diagram of a computer assisted medical procedure system 500 during a seventh step of a medical procedure according to one embodiment of the disclosure. Here, the captured x-ray images 580 may be relayed from the fluoroscopy device 540-2 to the cloud web service 510. The cloud web service 510 may relay these x-ray images 580 with the overlayed patient fiducials thereon to the medical server 525 and to the registering computing device 520 and head-mounted display device 515. The registering computing device 520, an operatively-coupled server, or both may be located within the medical procedure theater 530 with the patient and medical personnel 555-1, 555-2 for the medical personnel 555-1, 555-2 to see, in real time, the relative position of the needle 540-1 and fluoroscopy device 540-2 relative to the patient based on the processing of the data described herein. The head-mounted display device 515 may also be used similarly to identify the location of these devices relative to the patient during the medical procedure. Still further, the head-mounted display device 515 may be used, in an example embodiment, to overlay on top of a patient a three-dimensional image of the patient's anatomy (e.g., bones) over an image of the patient where the first medical device is a computed tomography (CAT) scan image that provides a real image of the patient's anatomy. This image may be overlayed on top of the image of the patient in real-time to also provide to the medical personnel 555-1, 555-2 the position of the patient's body relative to the fluoroscopy device 540-2 and/or needle 540-1.

The step in FIG. 17 may also include converting the x-ray images 580 (e.g., DICOM image files) into a PNG file or other appropriate file type. The conversion of the DICOM file to a PNG file may facilitate the use of the images in the overlay process and representation of these images at the registering computing device 520 and/or head-mounted display device 515. In an embodiment, a pixel-to-meter (PtM) ratio may be calculated. The PtM ratio may be descriptive of the distance of a pixel in meters thereby allowing the distances between the patient's anatomy and the needle 540-1 and/or fluoroscopy device 540-2 to be determined in real-time.

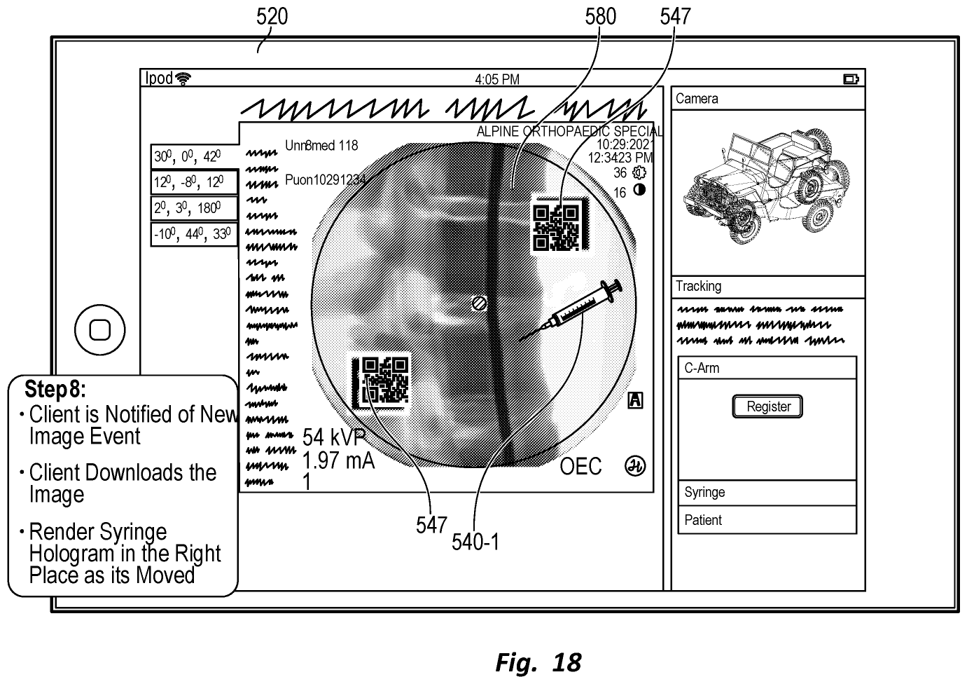

FIG. 18 is a graphical diagram of a computer assisted medical procedure system 500 during an exemplary eighth step of a medical procedure according to one embodiment of the disclosure. At FIG. 18, the x-ray images 580 may be rendered and presented to a display device such as the registering computing device 520 and/or head-mounted display device 515. In an embodiment, the registering computing device 520 may have served previously as the calibrating computing device 120 described in connection with FIG. 1. Therefore, in an embodiment, the registering computing device 520 may serve both of these purposes of calibrating the location of objects within the environment and used by the user to perform the medical procedures as described herein. The rendered x-ray images 580 may include the partially opaque fiducial 547 that may be seen by the medical personnel 555-1, 555-2 within the medical procedure theater 530 on the x-ray images 580 correlating to the fiducials 545 being placed on the patient's body.

As described herein, the movement of the needle 540-1 with its second medical device locator 535-2 may be detected by the calibrating computing device 520 or other movement detection device. It is appreciated that, although FIG. 18 shows a single needle 540-1, the present specification contemplates that the medical procedure may include the use of multiple needles or other medical devices that may also be concurrently tracked as described herein. The detection of the movement of the second medical device locator 535-2 allows the computer assisted medical procedure system 500 to determine the location of the needle 540-1 relative to the patient's anatomy and the partially opaque fiducials 547 thereon. When ready, the medical personnel 555-1, 555-2 may be notified of the x-ray images 580 captured and the location of the needle 540-1 relative to the patient's device. This x-ray image 580 may be automatically relayed to the registering computing device 520 or may be downloaded to the registering computing device 520 when the medical personnel 555-1, 555-2 is notified of the availability of the new x-ray images 580.

As the medical personnel 555-1, 555-2 bring the needle 540-1 closer to the patient's anatomy, a hologram or overlayed image of the needle 540-1 may be represented over the x-ray images 580. This image of the needle 540-1 may be updated in real time so that the medical personnel 555-1, 555-2 is updated on the location of the needle 540-1 relative to, in this example embodiment, the spine of the patient.

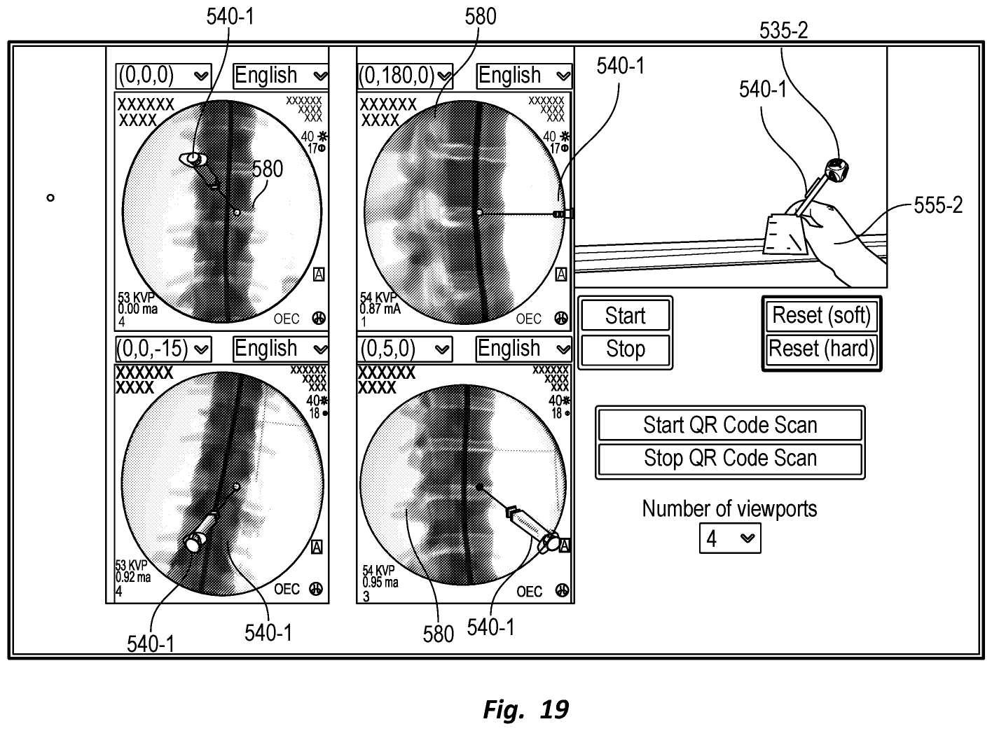

FIG. 19 is a graphical diagram of a GUI associated with the operation of the computer assisted medical procedure system 500 during the eight step of a medical procedure according to one embodiment of the disclosure. Similar to FIG. 18, FIG. 19 shows four distinct images, as shown in FIG. 19, of the patient's anatomy with the needle 540-1 image overlayed on top of these images. It is appreciated that more or less than four images may be presented to the user showing the patient's anatomy with the needle 540-1 image overlayed on top of these images. These four images allows a medical personnel 555-1, 555-2 to adjust the needle 540-1 relative to the patient's body. These four x-ray images 580 allow the medical personnel 555-1, 555-2 to determine whether the needle 540-1 should be moved left, right, up, down, in, or out from the patient's anatomy because the image of the needle 540-1 relative to the patient's anatomy is updated in real time.

FIG. 19 further shows a real image of the medical personnel 555-2 holding the needle 540-1 with its second medical device locator 535-2 attached thereto. As the medical personnel 555-2 moves the needle 540-1, the image of the needle 540-1 on the four x-ray images 580 also moves showing the relative position of the needle 540-1 to the patient's anatomy.

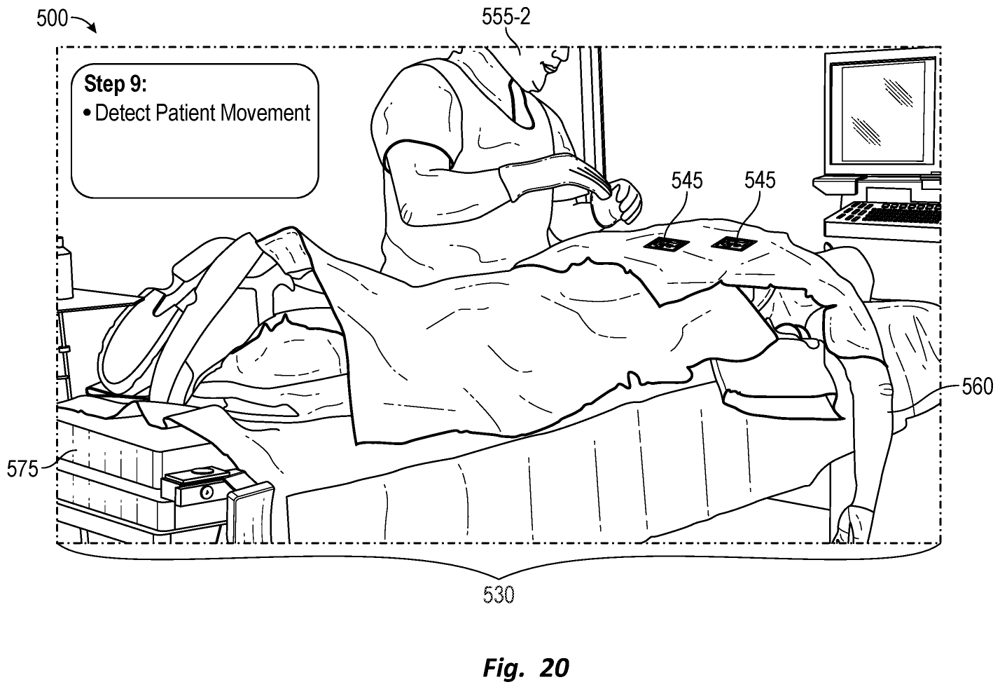

FIG. 20 is a graphical diagram of a computer assisted medical procedure system 500 during a ninth step of a medical procedure according to one embodiment of the disclosure. The ninth step described herein may describe the observance, at any time, of movement of the patient 560 in order to accommodate for such movement during the processes described herein. Movement of the patient may alter the representation of the image of the needle 540-1 relative to the patient's anatomy as shown in FIGS. 18 and 19. However, with the inclusion of the partially opaque fiducial 545 and the second medical device locator 535-2 on the needle 540-1, the relative position of the needle 540-1 to the patient's anatomy does not change because these are the fiducial markers that are being detected by the registering computing device 520 or other position location device within the medical procedure theater 530. In an embodiment, where movement of the patient 560 is detected, the medical personnel 555-2 may take new x-ray scans as described herein. Therefore, although the patient may shift his or her weight on the operation table 575, the relative location of the needle 540-1 to the patient's anatomy (e.g., due to the patient fiducials 545) does not change and the medical personnel 555-1, 555-2 may continue with the medical procedure (e.g., a steroid injection) confident that the position of the needle 540-1 is accurately represented on the x-ray images 580 display to the medical personnel 555-1, 555-2.

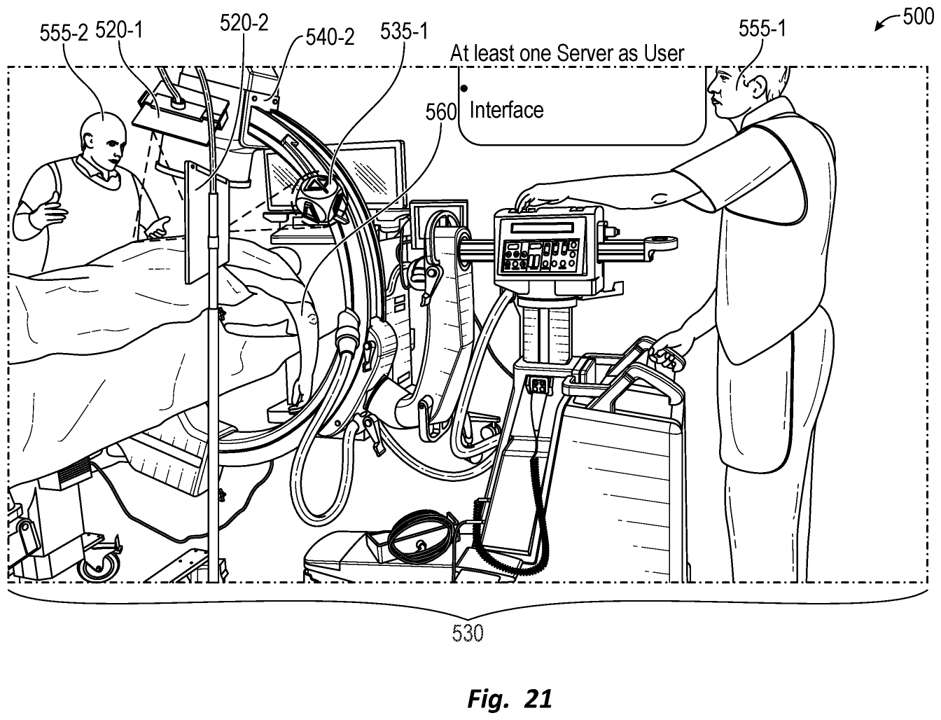

FIG. 21 is a graphical diagram of a computer assisted medical procedure system 500 during a tenth step of a medical procedure according to one embodiment of the disclosure. FIG. 21 shows that multiple registering computing devices 520-1, 520-2 may be used by the medical personnel 555-1, 555-2 to monitor the relative position of the needle 540-1 and/or fluoroscopy device 540-2 relative to the patient's 560 anatomy. In an embodiment, a first registering computing device 520-1 may be used to represent a first or first set of x-ray images 580 overlayed with an image of the needle 540-1 while the second registering computing device 520-2 is used to represent a second or second set of x-ray images 580 overlayed with the needle 540-1 image similar to those x-ray images 580 depicted in FIG. 19. FIG. 21 shows that multiple devices with multiple cameras may be used to detect the positions of the medical devices described herein as, in an example embodiment, a redundancy process to detect and update the locations of the medical devices.

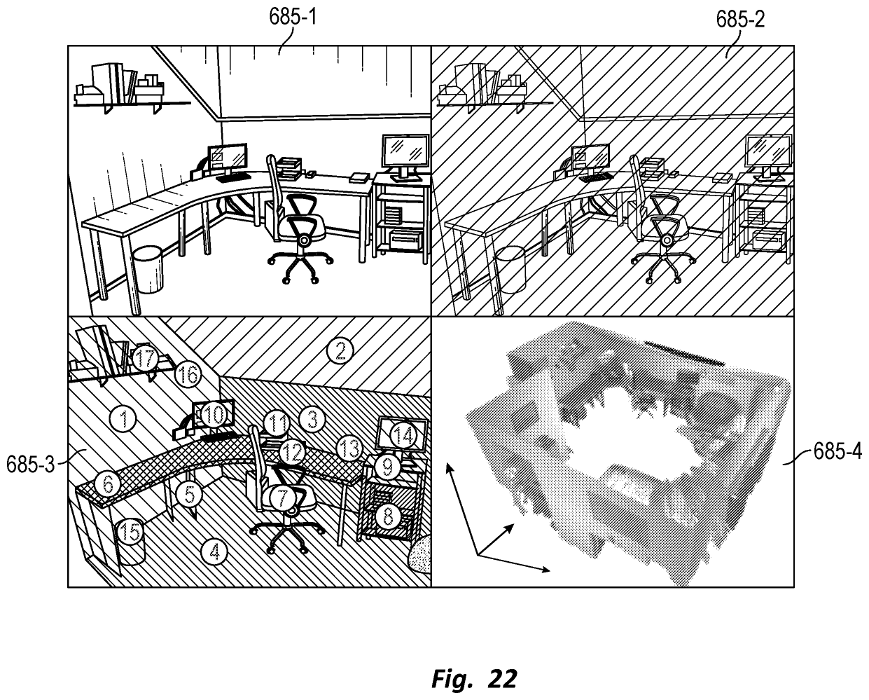

FIG. 22 is a diagram of a plurality of spatial object location images 685-1, 685-2, 685-3, 685-4 for a computer assisted medical procedure system according to one embodiment of the disclosure. A first spatial object location image 685-1 may be a camera image of an environment in which the head-mounted display device described herein is located. A second spatial object location image 685-2 may be a black and white, rasterized image of the environment that the head-mounted display device is located within. A third spatial object location image 685-3 may be an object identification image of the environment that the head-mounted display device is within indicating the distinct objects identified from the second spatial object location image 685-2. Additionally, a fourth spatial object location image 685-4 may be presented to a user of the head-mounted display device that successfully represents a three-dimensional image of the room in with the head-mounted display device is located within.

In an embodiment, the spatial object location images 685-1, 685-2, 685-3, 685-4 are obtained by a head-mounted display device that uses an onboard imaging device to render these images in order to present to the user a three-dimensional image of the room along with other objects therein including the registering computing device 620, the fluoroscopy device (e.g., 540-2 described herein) with its first medical device locator 635-1, the needle 640-1 with its second medical device locator 635-2, the x-ray images (e.g., 580 described herein) generated via the fluoroscopy device, the medical personnel (e.g., 555-1, 555-2 described herein), among other objects. This rendering allows for the user such as one of the medical personnel to wear the head-mounted display device in order to experience an augmented reality environment when conducting the medical procedure. In an embodiment, the head-mounted display device may be an inward-out head-mounted display device that does not require outside image detectors or sensors to compile the images presented in FIG. 22. In another embodiment, the head-mounted display device may be an outward-in head-mounted display device that relies on external cameras or sensors to compile the images presented in FIG. 22.

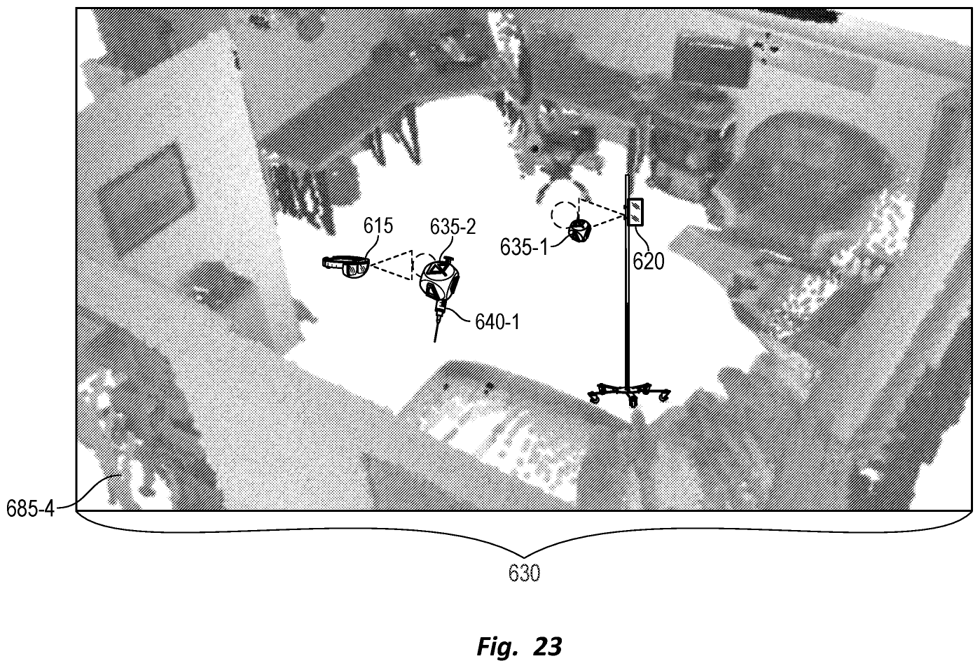

FIG. 23 is a diagram of a spatial object location images 685-4 with hardware associated with a computer assisted medical procedure system according to one embodiment of the disclosure. As described herein, the head-mounted display device 615 and/or registering computing device 620 may be used to determine the location of the fluoroscopy device (not shown) with its first medical device locator 635-1 and the needle 640-1 with its second medical device locator 635-2 within a medical procedure theater 630.

In an embodiment, the location of the registering computing device 620 may be set to 0,0,0 in Cartesian coordinates with the first medical device locator 635-1 and second medical device locator 635-2 set to coordinates that are based away from the registering computing device 620. Additionally, or alternatively, the location of the head-mounted display device 615 may be set to 0,0,0 in Cartesian coordinates with the first medical device locator 635-1 and second medical device locator 635-2 set to coordinates that are based away from the head-mounted display device 615. With these detected coordinates and the detected coordinates of the patient fiducials described herein, the relative positions of the needle 640-1 and fluoroscopy device relative to the patient may be determined. Still further, with these distances and the determined PtM distances, the location of the needle 640-1 relative to the patient may be accurately depicted in the x-ray images as described herein.

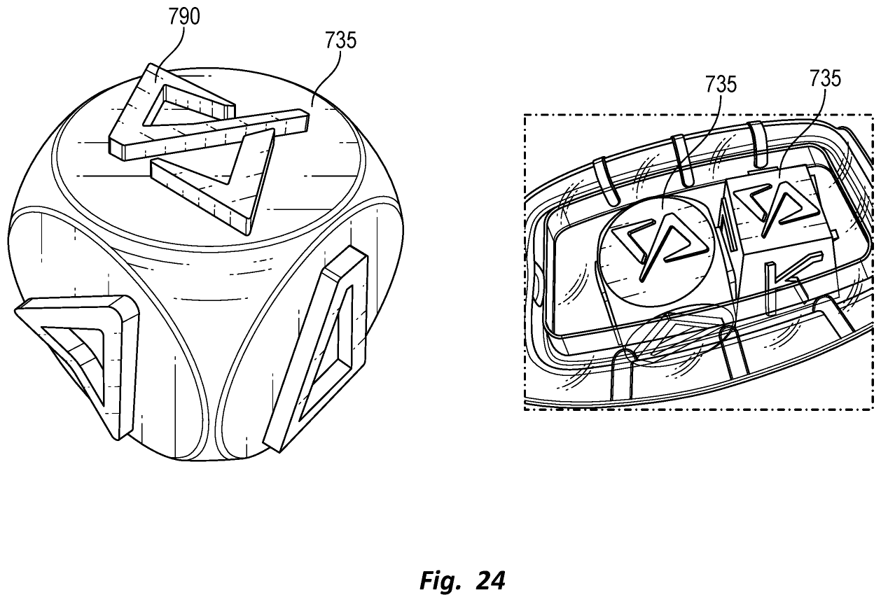

FIG. 24 is a diagram of a plurality of medical device locators 735 for a computer assisted medical procedure system according to one embodiment of the disclosure. As described herein, the example medical device locators 735 may be attached to any medical device used during a medical procedure such as a fluoroscopy device or a needle. It is appreciated that other types of medical device locators 735 may be used in the processes described herein and the medical device locators 735 shown in FIG. 24 are merely examples. The medical device locators 735 may each have six sides that are distinct from each other so that any given side of the medical device locators 735 may be distinguishable from each other thereby notifying the registering computing device and/or head-mounted display device of the orientation of the medical device locators 735 and, accordingly the orientation of the medical devices to which they are attached. In the example embodiment shown in FIG. 24, the distinctness of each side may be rendered by a raised topography 790. The registering computing device and/or head-mounted display device may be provided with a description of the raised topography 790 so that subsequent imaging of the medical device locators 735 may be used to identify the orientation of the medical device locators 735.

The present specification describes a computer-assisted surgical system that includes multiple tracking devices to direct medical personnel within a medical procedure theater to correctly perform that medical procedure. To track the medical instruments (e.g., needles and fluoroscopy devices for example), various tracking targets such as the fiducials described herein may be used to track both the medical devices as well as individual targets such as the patient. In order to track these medical devices, multiple tracking devices such as multiple cameras or tablet devices having cameras may be used. By having multiple tracking devices rather than a single tracking device, increased accuracy and a larger tracking area may be realized. These tracking devices may use any method to determine their relative locations within the area or, in the examples presented herein, within the medical procedure theater.

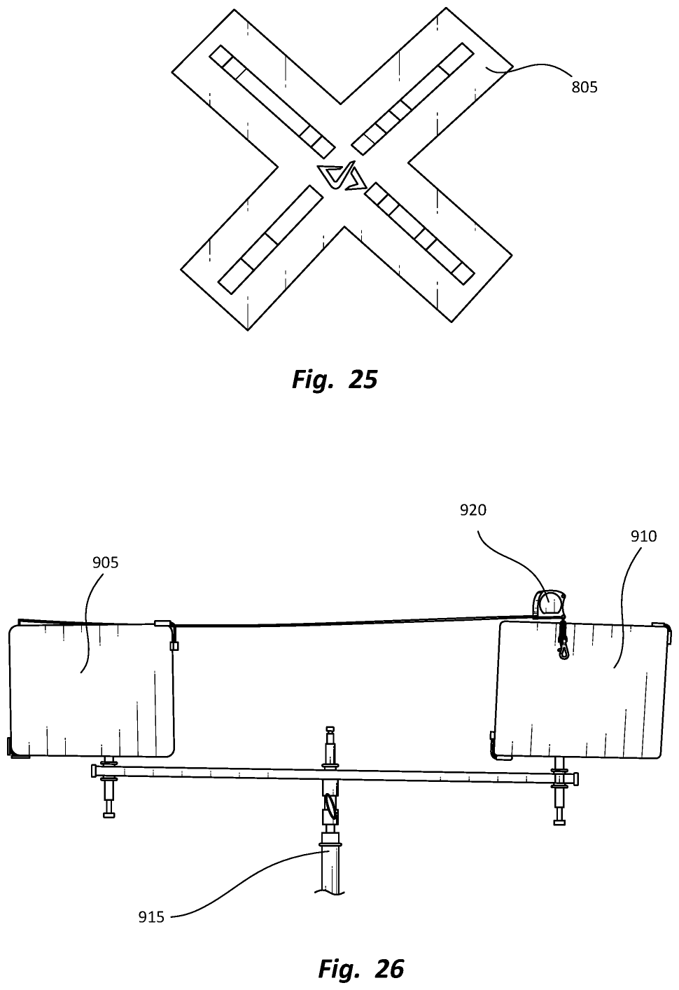

Turning to FIG. 25, a spatial anchor may be used to serve as a point in three-dimensional (3D) space that is viewable or within a line of sight of each of the devices. As described herein in some embodiments, the computer assisted medical procedure system may include multiple devices with some used to track targets, medical device locators, or fiducials while other devices are used by medical professionals to guide the medical professionals during the medical procedure. It is appreciated that those devices that track other devices can serve to guide the medical professionals during the medical procedure as well. It is also appreciated that these devices may be used to also calibrate medical devices and their locations within a medical procedure theater and, once the calibration process is completed, serve to track other devices and/or guide the medical professionals during the medical procedure. Where multiple devices are used, increased accuracy in both the calibration processes described herein as well as the execution of the medical procedure may be realized. Additionally, with a plurality of devices being used, the tracking area may also be increased. However, with the introduction of a plurality of devices used to track other devices or assist in providing guidance during the medical procedure, the specific location of each device relative to the other devices must be known. As such additional device calibrations may be completed in order to define the locations of each device with the medical procedure theater prior to the medical professionals initiating the medical procedure.

One method of allowing for multiple devices to determine their relative position to each other is to use a spatial anchor 805 within the medical procedure theater as shown in FIG. 25. The spatial anchor 805 may be placed within the medical procedure theater that is within the line of sight of all detecting devices (e.g., cameras or other optical devices). In an embodiment, the spatial anchor 805 may include a point cloud that is a set of data points in 3D coordinate system each representing a single spatial measurement on an object's surface such as those surfaces within the medical procedure theater. The use of the spatial anchor 805 allows each device to know their relative positions to the spatial anchor 805 and to each other as a result in an exemplary embodiment. It is appreciated that in those computer assisted medical procedure systems described herein where a plurality of devices are used to track other devices or assist in providing guidance during the medical procedure, any number of spatial anchors 805 may be placed within the medical procedure theater.

In an embodiment, these devices that are used to track other devices or assist in providing guidance during the medical procedure may include at least two cameras affixed to a support structure such as a cross bar or other stabilizing structure that positions each of the cameras at a set location relative to each other. This arrangement is shown in FIG. 26.

FIG. 26 is a diagram of a first image capturing device 905 and a second image capturing device 910 operatively coupled to a support structure 915 according to an embodiment. As described herein, the first image capturing device 905 and second image capturing device 910 may be used to detect a location of a spatial anchor (not shown) within a medical procedure theater in order to determine the location of each of the first image capturing device 905 and second image capturing device 910 within that medical procedure theater.

The embodiment shown in FIG. 26 shows that the first image capturing device 905 and second image capturing device 910 are fixed to the support structure 915 such that the distance between the first image capturing device 905 and second image capturing device 910 (e.g., a camera formed within a tablet-type computing device) are a set distance and location away from each other. In an embodiment, in order to calibrate the distance between the first image capturing device 905 and the second image capturing device 910 may include a technician, a medical professional, or other user of the computer assisted medical procedure system to use a tape measure 920 to physically measure the distance between the first image capturing device 905 and second image capturing device 910. This physical distance between the first image capturing device 905 and second image capturing device 910 may be input at, for example, the medical server. Because the dimensions of the support structure 915 are known and can be provided to the medical server, the medical server may calculate the relative position of the first image capturing device 905 to the second image capturing device 910. In an embodiment, the relative rotational positions of the first image capturing device 905 to the second image capturing device 910 may also be determined where the support structure 915 allows for such rotational movement of the first image capturing device 905 and second image capturing device 910 while attached to the support structure 915. In this embodiment, other calculations may be made by the medical server or other computing device as described herein.

Once this relative positioning of the first image capturing device 905 to the second image capturing device 910 is determined, the medical server may be used to calculate or triangulate the position of the spatial anchor (e.g., spatial anchor 805 in FIG. 25) within the medical procedure theater and, therefore, the relative position of the first image capturing device 905 and second image capturing device 910 to the spatial anchor. This calculation may be made in real time such that movement of the support structure 915 can be accounted for so that the real-time positions of the first image capturing device 905 and second image capturing device 910 may be known throughout the calibration process as well as throughout a medical procedure. As described herein, the determination of the first image capturing device 905 and second image capturing device 910 also allows for the medical server to receive captured images to perform a triangulation of a variety of extrinsic points and/or the spatial anchors in order to determine the position of the first image capturing device 905, the second image capturing device 910, and the support structure 915 within the medical procedure theater in order to use the first image capturing device 905 and second image capturing device 910 in determining, for example, the relative position of a first medical device to a second medical device and these two medical devices relative to a patient's anatomy.

In an embodiment, the medical server or other computing device associated with the computer assisted medical procedure system described herein may determine the relative positions of the first image capturing device 905 and second image capturing device 910 within the medical procedure theater acting as a stereoscopic camera system. The relative positions of the first image capturing device 905 and the second image capturing device 910 within the medical procedure theater may be calculated by executing, for example, an eight-point algorithm via a hardware processing device at the medical server or other computing device. The eight-point algorithm may be used in computer vision to estimate an essential matrix or a fundamental matrix that define the positions of the first image capturing device 905 and second image capturing device 910 from a set of corresponding image points or camera extrinsics. The camera extrinsics can then be calculated from the fundamental matrix estimated via the eight-point algorithm. In an embodiment, the eight-point algorithm requires at least eight corresponding points from each two-dimensional (2D) image from the stereo camera pair that comprise the first image capturing device 905 and second image capturing device 910.

In order to calculate the camera extrinsics, the first image capturing device 905 and second image capturing device 910, as the stereo camera pair, may be configured to track multiple targets that may include, for example, a spatial anchor, fiducials, edges of a target, etc. In an embodiment, a 3D box with visual targets on them may be used to supply the targets that each of the first image capturing device 905 and second image capturing device 910 may capture and the medical server may identify in the respective 2D images. In the example embodiment, where the target includes a 3D box, at least eight known offsets from an identified center point (e.g., such as the eight corners of the 3D box) such that, while tracking, an image analyzer at the medical server may identify the 3D position of nine corresponding points (e.g., including the center point) at that target. Where these 3D box targets are placed on a medical device, for example, the 3D position of the medical device may be determined. Thus, by using the known camera intrinsics (e.g., focal length, aperture, field-of-view, resolution, etc.), nine corresponding 3D points may be converted into nine corresponding 2D points on the 2D captured images which represent the pixel location (e.g., not an integer) of each point in the 2D camera image. If four of these 3D boxes are used, 36 corresponding points may be identified within each 2D image captured by each of the first image capturing device 905 and second image capturing device 910. This amount of data may be sufficient to calculate the camera extrinsics as described herein.

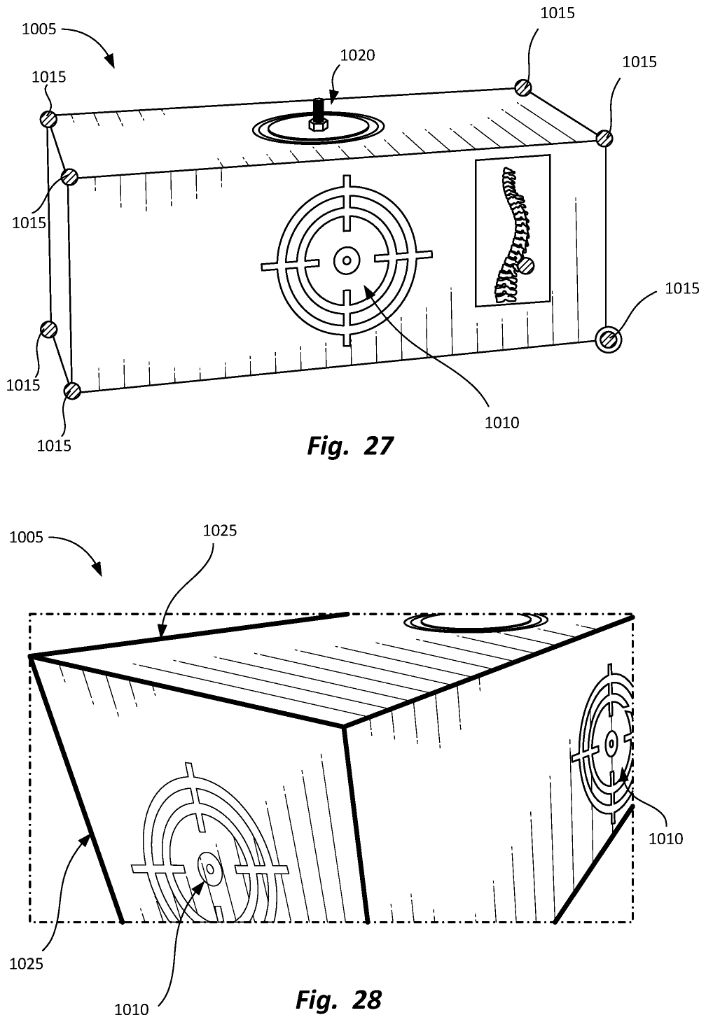

FIG. 27 is a diagram of a 3D box target 1005 used as a visual target for a stereoscopic camera system according to an embodiment. As described herein, the 3D box target 1005 may be operatively coupled to any medical device, for example, within the medical procedure theater which may include a fluoroscopy device, an operation table, or any other medical device. In an embodiment, mounting hardware 1020 may be used to secure the 3D box target 1005 to the medical device.

As described herein, the 3D box target 1005 has a 3D shape such as a box shape such that two or more edges of the box meet together to form extrinsic points 1015. In the example presented in FIG. 27, the number of potential extrinsic points 1015 within a field of view of either of the first or second image capturing devices (e.g., 905, 910 in FIG. 26). Although some extrinsic points 1015 may not be detected within the individual 2D images by the medical server after receiving those 2D images captured by the first and second image capturing devices, these extrinsic points 1015 may still be used to detect the position of the 3D box target 1005 within the medical procedure theater and, thus, a position of the medical device the 3D box target 1005 is coupled to. This is done by triangulating two known extrinsic points 1015 within the same coordinate space. In an embodiment, part of the triangulation algorithm executed by a hardware processor of the medical server or other computing device includes calculating an outline of the 3D box target 1005 (e.g., such as drawing a box based on the eight corners, where all eight corners are calculated separately using triangulation). All of the angles of the outline of this 3D box target 1005 should be nearly 90 degrees. Likewise, the edges of the outline of the 3D box target 1005 should have corresponding lengths of the same distance. In an embodiment, the variation of the length of the edges and the angles of each corner (e.g., where the 3D box target 1005 is a rectangular cuboid) serve as an indication of accuracy of the camera extrinsics. The more accurate the camera extrinsics are, the more accurate the detected edges and angles are. Thus, when calculating the camera extrinsics from various view samples, the hardware processor of the medical server compute which camera extrinsics values will produce the most accurate 3D box target 1005 outlines. This accuracy is referred to as the triangulation accuracy estimation and can be used to validate the calculated camera extrinsics.

In an embodiment, a center point 1010 serving as a ninth camera extrinsic may be visually placed on a surface of the 3D box target 1005. This center point 1010 may be detectable within any 2D image captured by the first or second image capturing devices. With the hardware processor, the center point 1010 may be detected and used as this ninth camera extrinsic in those instances where, for example, a corner on the 3D box target 1005 is not within a line of sight of one or both of the first or second image capturing devices due to its orientation relative to the first or second image capturing devices.

FIG. 28 is a diagram of a 3D box target with edges 1025 highlighted by a hardware processing device of a medical server indicating an outline of the 3D box target 1005 according to an embodiment. As described herein, the 3D box target 1005 includes a center point 1010 used as an extrinsic point where, in the example shown in FIG. 28, one or more corners of the 3D box target 1005 are not viewable from certain angles.

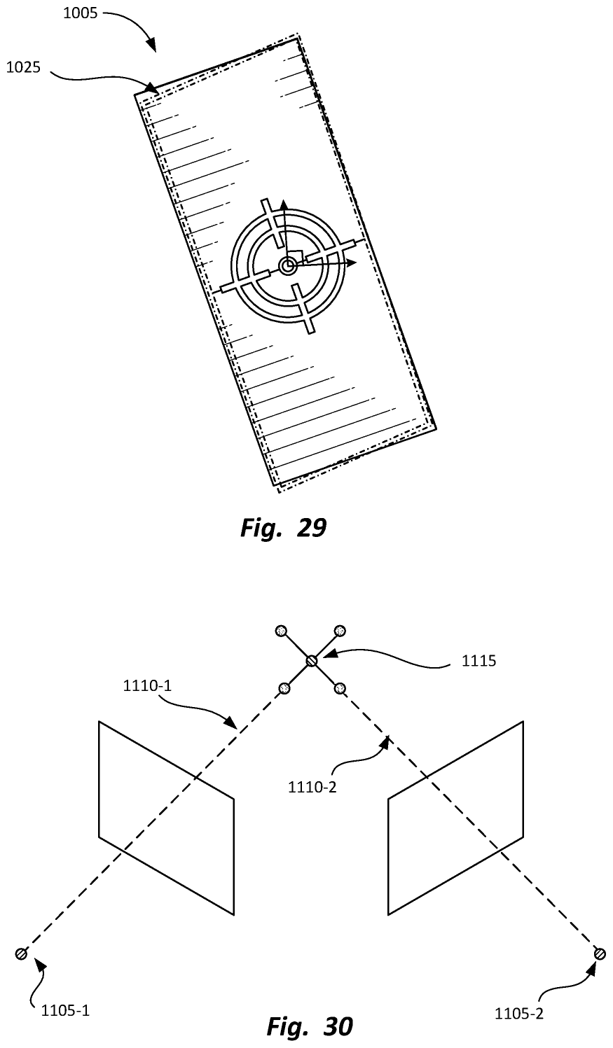

By detecting the extrinsic points as shown in FIG. 27, the edges 1025 of the 3D box target 1005 may be extrapolated. These lines that define the edges 1025 in a first 2D image received by the medical server from the first image capturing device may not necessary match similar lines defined in a second 2D image received by the medical server from the second image capturing device. FIG. 29 shows this ambiguity in these defined edges 1025.

FIG. 29 is a diagram of a 3D box target 1005 with detected edges 1025 highlighted by a hardware processing device of a medical server indicating an outline of the 3D box target 1005 according to an embodiment. In this example embodiment, an offset between the detected edges 1025 and the actual edges of the 3D box target 1005 is detected by the hardware processor of the medical server executing the eight-point algorithm described herein. The hardware processor may detect the misalignment of the detected edges 1025 with the actual edges of the 3D box target 1005 indicating a level of inaccuracy between the detected extrinsic points described herein. This overlay of the detected edges 1025 on the 3D box target 1005 may be completed by the hardware processor projecting a position generated by the first image capturing device into the coordinate space of the second image capturing device and visa-versa. In order to increase the accuracy of detection of the orientation of the 3D box target 1005, the hardware processor may execute a triangulation algorithm.

FIG. 30 is a block diagram showing a triangulation process of triangulating a 3D box target within a 3D space according to an embodiment. Triangulation is the process of refining a detected 3D position of, for example, the 3D box target and the extrinsic points by using data from both the first image capturing device 1105-1 and second image capturing device 1105-2 described herein. The two detected physical positions of the 3D box target by the first image capturing device 1105-1 and second image capturing device 1105-2 are combined using the camera extrinsics for greater accuracy during tracking. The first image capturing device 1105-1 and second image capturing device 1105-2 each independently track the 3D box target's position 1115 in its own coordinate space, but using the camera extrinsics, the hardware processor of the medical server may project a position generated from the image received by the first image capturing device 1105-1 into the coordinate space of the image captured by the second image capturing device 1105-2 and visa-versa. This produces two relatively close 3D position values for a given point in the same coordinate space. However, rather than average the X, Y, Z coordinate values of the point in those position values, each point is recognized as most likely inaccurate by its distance from the viewpoint of the first image capturing device 1105-1 and second image capturing device 1105-2. In an embodiment, if there was a first line of sight 1110-1 from a camera lens of the first image capturing device 1105-1 or a second line of sight 1110-2 from a camera lens of the second image capturing device 1105-2 to a specific 3D point (e.g., an extrinsic points) the “real” or actual point on the 3D box target's position 1115 is likely located on that line of sight 1110-1, 1110-2. Similarly, other positions may be translated from the other of the two first or second image capturing devices 1105-1, 1105-2. Because these two lines of sight 1110-1, 1110-2 are not parallel they “cross” in 3D space and they may not actually touch. If they did touch, then the point where they intersect is the more accurate 3D special location of the 3D box target's position 1115. The two non-parallel first line of sight 1110-1 and second line of sight 1110-2 which may not actually touch each other have a point on each line which are closest to each other. In an embodiment, the hardware processor of the medical server may calculate those two points (one on each of the first line of sight 1110-1 and second line of sight 1110-2) and average of them. The result is a new triangulated point for the 3D box target's position 1115.

For greater accuracy, in an embodiment, each of the eight corner points or extrinsic points of the 3D Box target may be projected separately. The hardware processor of the medical server may then calculate the final position (e.g., location and rotation) from those eight triangulated extrinsic points. The location of the center point (e.g., 1010 in FIG. 27) is calculated as an average of those extrinsic points.

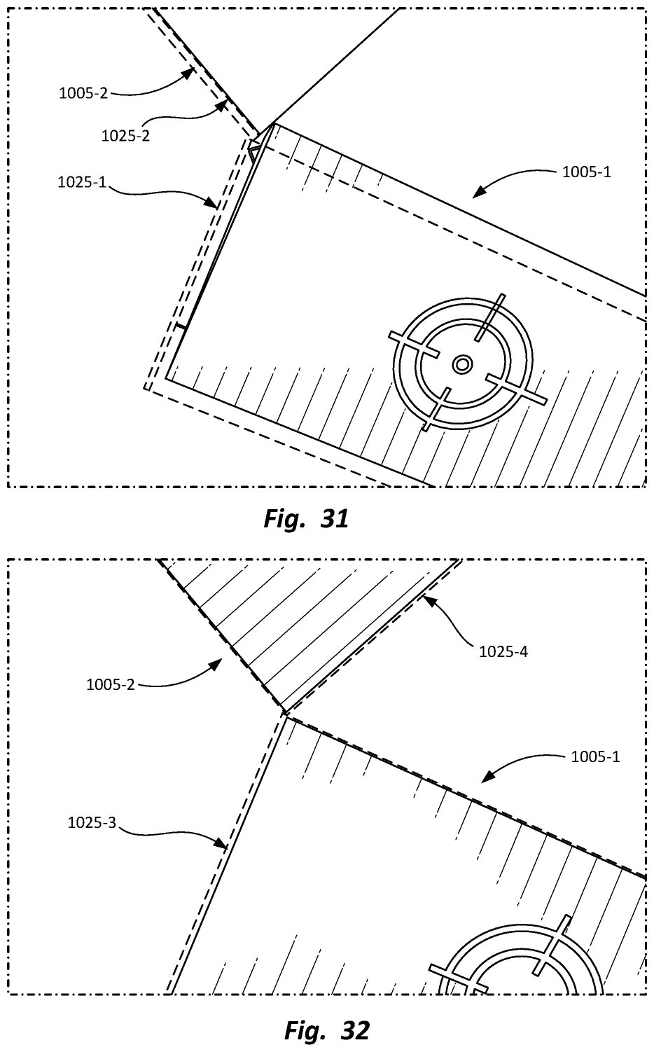

The rotation/quaternion may be calculated by treating the triangulated corners of the 3D box target as the corners of a digital box with six sides. Each side of this digital box is defined by four points, but they may not lie perfectly on the same plane as shown in FIG. 29, for example. Instead, by calculating the location of the four planes for each side separately, this rotation/quaternion may be calculated. For example, if the top side of the 3D box target has points A, B, C, and D, the hardware processor of the medical server can calculate four planes of this digital box with each combination of three points (ABC, ABD, BCD, ACD). The hardware processor then computes a vector normal for each of those four planes and averages them together. By doing this with the top, bottom, front, and back of the 3D box target, the four vector normals that represent the rotation of the 3D box target are also determined. By averaging the top/bottom and front/back and using those two remaining Y and Z vectors, the rotation of the digital box may be calculated. This process is further shown in FIGS. 31 and 32.

FIG. 31 is a diagram of a first 3D box target 1005-1 and second 3D box target 1005-2 with first detected edges 1025-1 and second detected edges 1025-2, respectively, as indicated by a hardware processing device of a medical server indicating an outline of the first 3D box target 1005-1 and second 3D box target 1005-2 according to another embodiment. FIG. 31 shows an outline from the execution of the eight-point algorithm that defines first detected edges 1025-1 that may not match with each other. Further, FIG. 31 also shows a second 3D box 1005-2 that is within the field of view of the first image capturing device and second image capturing device as described herein which also includes second detected edges 1025-2 defined after execution of the eight-point algorithm. Additionally, FIG. 32 is a diagram of a first 3D box target 1005-1 with derived edges 1025-3 and a second detected edges 1025-2 with derived edges 1025-4 highlighted by a hardware processing device of a medical server according to another embodiment. The second 3D box target 1005-2 also includes highlighted second detected edges 1025-2.

As described herein, an offset between the detected edges 1025-1, 1025-2, and the actual edges of the first 3D box target 1005-1 and second 3D box target 1005-2 is detected by the hardware processor of the medical server executing the eight-point algorithm described herein. The hardware processor may detect the misalignment of the detected edges 1025-1, 1025-2 with the actual edges of the first 3D box target 1005-1 and second 3D box target 1005-2 indicating a level of inaccuracy between the detected extrinsic points shown in FIG. 31. In order to increase the accuracy of detection of the orientation of the first 3D box target 1005-1 and second 3D box target 1005-2, the hardware processor may execute a triangulation algorithm.

This triangulation process includes refining a detected 3D position of, for example, the first 3D box target 1005-1 and second 3D box target 1005-2 and the extrinsic points by using data from both the first image capturing device and second image capturing device described herein. The two detected physical positions of the first 3D box target 1005-1 and second 3D box target 1005-2 by the first image capturing device and second image capturing device are combined using the camera extrinsics for greater accuracy during tracking. The first image capturing device and second image capturing device each independently track the position of the first 3D box target 1005-1 and second 3D box target 1005-2 in their own coordinate space, but using the camera extrinsics, and the hardware processor of the medical server may project a position generated from the image received by the first image capturing device into the coordinate space of the image captured by the second image capturing device and visa-versa. This produces two relatively close 3D position values for a given point in the same coordinate space. However, rather than average the X, Y, Z coordinate values of the point in those position values, each point is recognized as most likely inaccurate by its distance from the viewpoint of the first image capturing device and second image capturing device. In an embodiment, if there was a first line of sight from a camera lens of the first image capturing device or a second line of sight from a camera lens of the second image capturing device to a specific 3D point (e.g., an extrinsic points) the “real” or actual point on the position of the first 3D box target 1005-1 and second 3D box target 1005-2 is likely located on that line of sight. Similarly, other positions may be translated from the other of the two first or second image capturing devices. Because these two lines of sight are not parallel, they “cross” in 3D space and they may not actually touch. If they did touch, then the point where they intersect is the more accurate 3D special location of the position of each of the first 3D box target 1005-1 and second 3D box target 1005-2. The two non-parallel first line of sight and second line of sight may not actually touch each other have a point on each line which are closest to each other. In an embodiment, the hardware processor of the medical server may calculate those two points (one on each of the first line of sight and second line of sight) and average of them. The result is a new triangulated point for the position of the first 3D box target 1005-1 and second 3D box target 1005-2.

For greater accuracy, in an embodiment, each of the eight corner points or extrinsic points of the first 3D box target 1005-1 and second 3D box target 1005-2 may be projected separately. The hardware processor of the medical server may then calculate the final position (e.g., location and rotation) from those eight triangulated extrinsic points. The location of the center point (e.g., 1010 in FIG. 27) is calculated as an average of those extrinsic points.

The rotation/quaternion may be calculated by treating the triangulated corners of the each of the first 3D box target 1005-1 and second 3D box target 1005-2 as the corners of a digital box with six sides. Each side of this digital box is defined by four points, but they may not lie perfectly on the same plane. Instead, by calculating the location of the four planes for each side separately, this rotation/quaternion may be calculated. For example, if the top side of each of the first 3D box target 1005-1 and second 3D box target 1005-2 have points A, B, C, and D, the hardware processor of the medical server can calculate four planes of this digital box with each combination of three points (ABC, ABD, BCD, ACD). The hardware processor then computes a vector normal for each of those four planes and averages them together. By doing this with the top, bottom, front, and back of the 3D box target, the four vector normals that represent the rotation of the 3D box target are also determined. By averaging the top/bottom and front/back and use those two remaining Y and Z vectors, the rotation of the digital box may be calculated as indicating by the corners of the derived edges 1025-3, 1025-4 of the first 3D box target 1005-1 and second 3D box target 1005-2 more closely resembling reality as shown in FIG. 32.

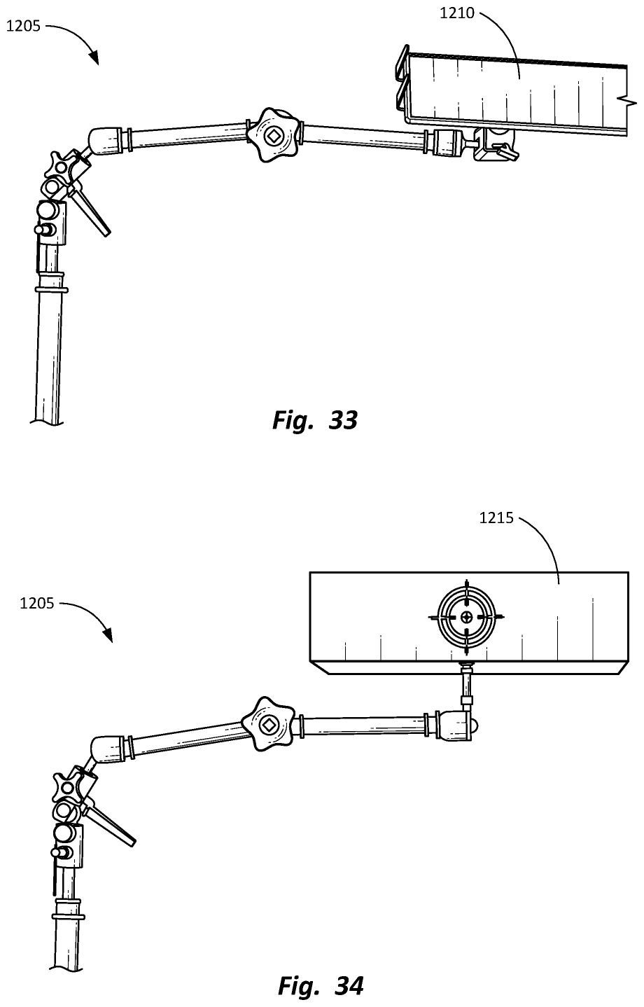

FIG. 33 is a subordinate device support structure 1205 used to hold a subordinate device used to track the location of a medical device in a medical procedure theater according to an embodiment. The subordinate device support structure 1205 holds a subordinate device that is being tracked, visually, by a primary device that visually tracks the subordinate device. In an embodiment, the subordinate device and/or primary device may include a plurality of devices such as cameras that capture a stereoscopic image if another device such as a medical device or the subordinate device. In FIG. 33, the subordinate device support structure 1205 includes a single location where a subordinate device may be held so that one or more primary devices can track the location of the single subordinate device.

As shown in FIG. 33, the subordinate device support structure 1205 includes a holding surface 1210. Similarly, FIG. 34 is a subordinate device support structure 1205 used to hold a subordinate device within a 3D box target 1215 used to track the location of a medical device in a medical procedure theater according to an embodiment. In an embodiment, the holding surface 1210 may be a flat surface into which the primary device is placed such that a camera of the primary device may capture images or video of a medical device within the medical procedure theater. The holding surface 1210 may include any fasteners such as clamps that hold the subordinate device in placed. The holding surface 1210 and fasteners may place the subordinate device in a specific location such that the camera of the subordinate device is not moved during use.

In an example, where a needle is being tracked (e.g., via Needle Tag) held by a physician, the subordinate device may be placed closer to the needle than the primary device. However, the subordinate device may be placed too close to the needle such that other medical devices such as a C-arm fluoroscopy device is not within the field of view of the camera of the subordinate device. In this example, therefore, the C-arm fluoroscopy device may be tracked by the primary device concurrently as the primary device also tracks the location of the subordinate device.

In an embodiment, the subordinate device can be tracked by the primary device by displaying an image target on a screen formed into the subordinate device (e.g., a tablet-type computing device). For example, a large tablet device (e.g., an Apple ° iPad®) can display the image target for tracking on a relatively large screen for the primary device(s) to view.

Without implementing the triangulation processes described herein, tracking an image target may not be as accurate as tracking a 3D box target in an embodiment. Thus, in an embodiment, a 3D box target 1215 such as those shown and described in FIGS. 27-29, 31, and 32 may be used. This 3D box target 1215 may be sufficiently large enough to contain the subordinate device within it and may include a cut-out for the camera of the subordinate device. Because the 3D box target 1215 has multiple sides with specified dimensions, the hardware processor executing the eight-point algorithm may use the plurality of sides of the 3D box target 1215 with which to use to compute the position of the 3D box target 1215 within the 3D space of the medical procedure theater. Whether the subordinate device is displaying an image target on its screen or is placed within the 3D box target 1215 as shown in FIG. 34, the target that a primary device will track may be referred herein as the subordinate device target in some embodiments.

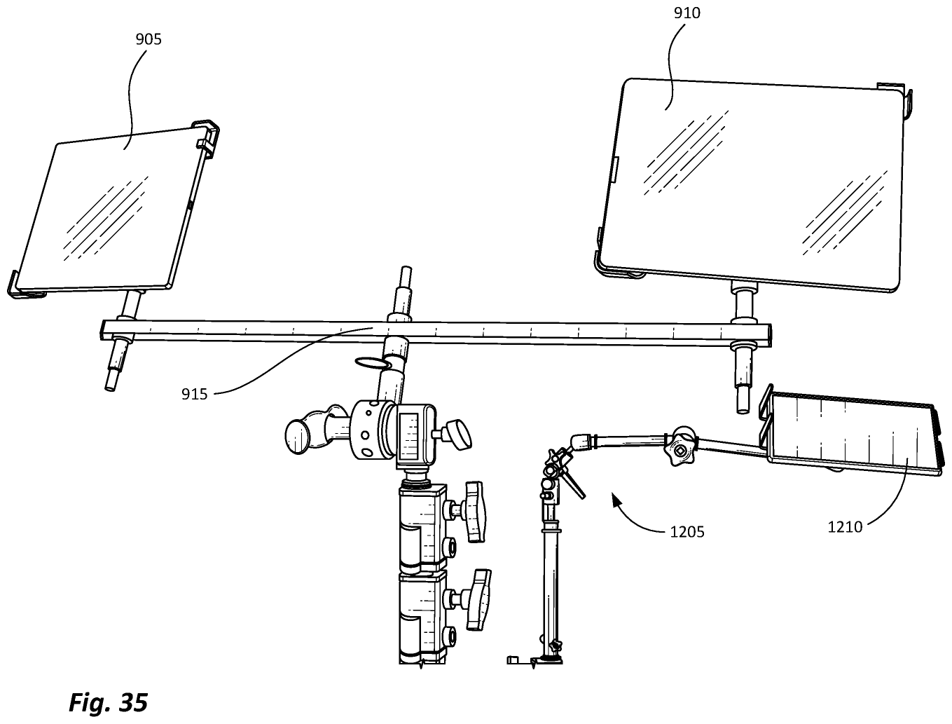

FIG. 35 is a diagram of a pair of primary devices consisting of a first image capturing device 905 and second image capturing device 910 relative to a holding surface 1210 for a subordinate device according to an embodiment. As described herein, the first image capturing device 905 and second image capturing device 910 may be used to detect a location of a spatial anchor (not shown) such as the 3D box target (e.g., 1215 of FIG. 34) placed on the subordinate device support structure 1205 within a medical procedure theater. In an embodiment, the first image capturing device 905 and second image capturing device 910 may be used to determine the location of each of the first image capturing device 905 and second image capturing device 910 within that medical procedure theater by tracking a spatial anchor (e.g., 805 of FIG. 25).

During operation, the offset from the subordinate device target (e.g., a target that a primary device will track such as the image displayed on the subordinate device or the 3D box target into which the subordinate device is placed) relative to the camera on the subordinate device may be determined. In an embodiment, the location of the subordinate device target is its center point such as the center point 1010 of FIG. 27 placed on a 3D box target or is a center of a display device display such as target displayed on the display device at the center of the subordinate device. In order to calculate the position of subordinate device target to be tracked by the cameras of the first image capturing device 905 and second image capturing device 910 in the coordinate space of the medical procedure theater, the distance between the first image capturing device 905 and second image capturing device 910 to the subordinate device target of the subordinate device may be determined. However, an offset value from the subordinate device target to the camera of the subordinate device may be a predetermined offset value based on the type of device used as the subordinate device and its intrinsic hardware characteristics. For example, the subordinate device may be an Apple ° iPad® that includes hardware dimensions and characteristics that define the offset value. In an embodiment, the medical server may be equipped to receive input describing the make, model, and manufacturer of the subordinate device such that when the make, model, and manufacturer data describing the subordinate device being placed in the holding surface 1210 of the subordinate device support structure 1205, the medical server may determine, from a device look-up table stored on a memory device of the medical server, the offset value between the camera of the subordinate device and the subordinate device target. This offset value may include x, y, and z coordinate values that indicate where the camera of the subordinate device is relative to the detected subordinate device target.

In an embodiment, the offset value may be calculated by a hardware processor of the medical server. This may be done where the subordinate device does not fit perfectly within the 3D box target 1215 when used. In this embodiment, the offset value may be calculated by the primary devices (e.g., the first image capturing device 905 and second image capturing device 910) and subordinate device capturing images in order to concurrently detect where a spatial anchor (e.g., 805 of FIG. 25) is within the medical procedure theater. By using the triangulation processes described herein, the medical server may track identical a spatial anchors (e.g., 805 of FIG. 25) with the subordinate device and primary devices log the position of a spatial anchor (e.g., 805 of FIG. 25) with the medical server. In an embodiment, the medical server may determine the relative position of the spatial anchor (e.g., 805 of FIG. 25) to each of the subordinate device and primary devices to calculate the offset value.

As described herein, along with the calibration and determination of the position of the subordinate device and primary devices, other medical devices being tracked by the subordinate device and primary devices may also be calibrated such at that described in connection with FIGS. 4 through 10. In the context of the present specification, this calibration may be conducted on a fluoroscopy device, a camera, a needle, and other devices used during the process described herein.

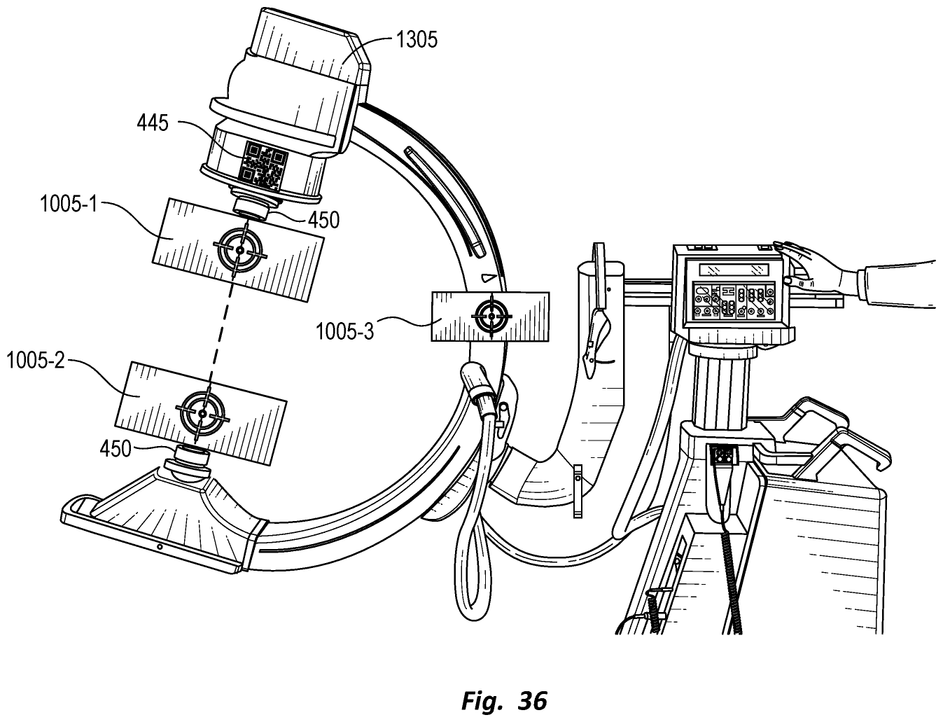

FIG. 36 is a diagram showing a c-arm shaped fluoroscopy device 1305 to be calibrated using a plurality of 3D box targets 1005-1, 1005-2, 1005-3 according to an embodiment. The fluoroscopy device 1305 may be similar to the fluoroscopy device described in connection with FIGS. 4-10 in an embodiment. By performing the calibration process on the fluoroscopy device 1305, the scanline or x-ray emissions from an x-ray emission node and an x-ray detection node.

As described herein, the fluoroscopy device 1305 may include a fiducial 445 similar to that described in connection with FIGS. 4-10. In this embodiment, a camera on the first image capturing device 905 or second image capturing device 910 of the primary device or a camera on the subordinate device may be used to scan the fiducial 445 on the fluoroscopy device 1305. This scan may identify the fluoroscopy device 1305 within a specific medical procedure theater, identify the specific type of device and qualities of the fluoroscopy device 1305, among other data associated with the operation of the fluoroscopy device 1305 within in the computer assisted medical procedure system described herein.

During the calibration processes of the fluoroscopy device 1305, device alignment caps 450 may be added to the fluoroscopy device 1305. In an embodiment, the device alignment caps 450 may be operatively coupled to an x-ray emission node and an x-ray detection node on the fluoroscopy device 1305. These device alignment caps 450 may allow a user to calibrate the fluoroscopy device 1305 prior to use on a patient during a medical procedure. The device alignment caps 450 may be added to the fluoroscopy device 1305 by medical personnel conducting the calibration of the fluoroscopy device 1305 in the medical procedure theater.

Along with the device alignment caps 450, a first 3D box target 1005-1 is placed at the x-ray emission node of the fluoroscopy device 1305, a second 3D box target 1005-2 is placed at an x-ray detection node of the fluoroscopy device 1305, and a third 3D box target 1005-3 is placed at a central location on the arm of the fluoroscopy device 1305 as shown in FIG. 36. The third 3D box target 1005-3 may be placed on the arm of the fluoroscopy device 1305 at a known location and may remain there so that as the fluoroscopy device 1305 is moved within the medical procedure theater after calibration so that the location of the x-ray emission node and x-ray detection node may be capable of being known. As a result of being capable of tracking each of the first 3D box target 1005-1, second 3D box target 1005-2, and third 3D box target 1005-3 simultaneously during the calibration process, the offset of the third 3D box target 1005-3 to the x-ray emission node and x-ray detection node is determined by the hardware processor of the medical server when the cameras of the primary device and/or subordinate device capture images of the fluoroscopy device 1305. The locations of the x-ray emission node and x-ray detection node are points with no rotation value. However, the scanline between the x-ray emission node and x-ray detection node does have a position which is the location of its center point and its rotation.

During the calibration process and while the primary devices and/or subordinate device are capturing images of the fluoroscopy device 1305 for the calibration, the medical server may identify the model of the fluoroscopy device 1305. In an embodiment, the model of the fluoroscopy device 1305 may be entered manually at the medical server using an input device such as a mouse or keyboard. In another embodiment, the DICOM data associated with the DICOM files during operation of the fluoroscopy device 1305 may indicate to the medical server the model of the fluoroscopy device 1305. Calculated calibration values, such as the distance between the x-ray emission node and the x-ray detection node are made to fit within thresholds that can vary with each fluoroscopy device 1305 model. For example, the OEC 9900® C-Arm fluoroscopy device by General Electric® may have a distance between the x-ray emission node and x-ray detection node larger or smaller than other models of fluoroscopy devices 1305. As a safeguard, the calibration process may only be allowed to proceed according to values approved for that model of fluoroscopy device 1305. Valid thresholds are stored as calibration values and include relative distances and relative rotations on a look-up table maintained on a memory device of the medical server. By storing these calibration values in the look-up table, the calibration process may be completed less frequently than would otherwise be required if these calibration values were not maintained. Because the third 3D box target 1005-3 placed on the arm of the fluoroscopy device 1305 may be placed at different locations on the arm of even identical fluoroscopy devices 1305, each fluoroscopy device 1305 will need to be calibrated prior to use by a medical professional. In an embodiment, when a fluoroscopy device 1305 creates a DICOM file, that fluoroscopy device 1305 has various DICOM tag values that may be used to uniquely identify the specific fluoroscopy device 1305.

As described herein, the calibration process of the fluoroscopy device 1305 may be completed by either or both of the cameras of the primary devices or subordinate device capturing an image of the first 3D box target 1005-1 and second 3D box target 1005-2 relative to the third 3D box target 1005-3. By executing the eight-point algorithm and triangulation algorithm described herein for each of the first 3D box target 1005-1, second 3D box target 1005-2, and third 3D box target 1005-3, the x-ray emission node and x-ray detection node may be derived by the hardware processor of the medical server thereby allowing the medical server to identify the location of the scanline during x-ray image capturing of the patient's anatomy.

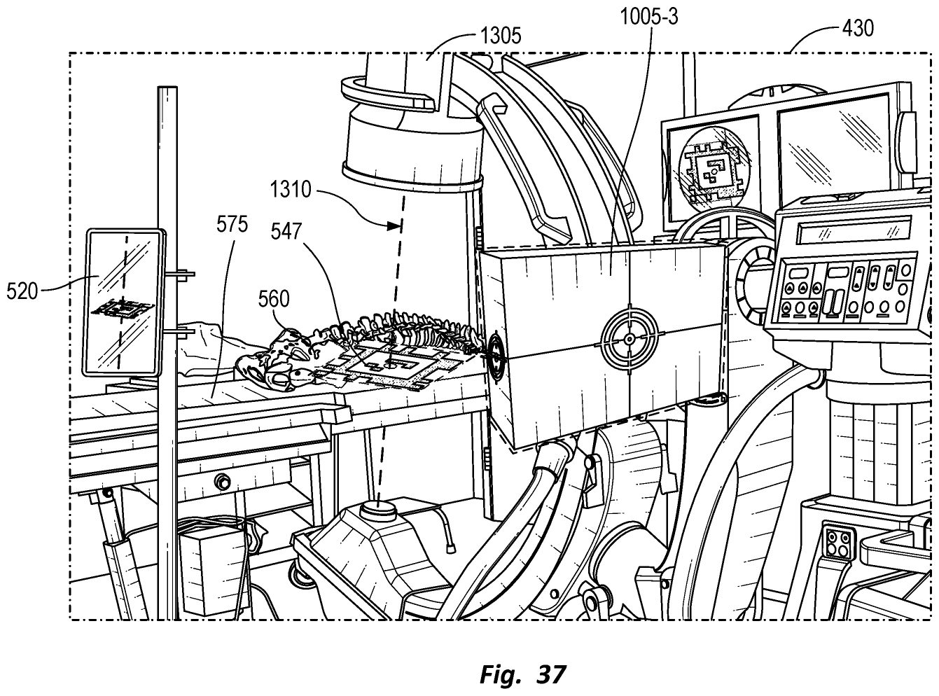

FIG. 37 is a diagram of a fluoroscopy device 1305 within a medical procedure theater 430 during a calibration check using a registering computing device 520 according to an embodiment. During this calibration check, the first 3D box target and second 3D box target described in FIG. 36 have been removed with the third 3D box target 1005-3 remaining on the arm of the fluoroscopy device 1305 for detection of the location of the scanline 1310 emitted from the x-ray emission node to the x-ray detection node.

The calibration check may include placing one or more partially opaque fiducials 547 onto am operation table 575 on or near a patient 560 surrogate. The primary device and subordinate device (not shown) may be used to monitor the orientation and position of the fluoroscopy device 1305 during this calibration check. While doing so, images are captured by a camera device of the subordinate device and/or primary device so that a representation of the scanline 1310 may be overlayed onto a DICOM image presented to a medical professional at a registering computing device 520 as described herein. In an embodiment, the medical professional may scan a partially opaque fiducial 547 such that the scanline 1310 is positioned directly in the center of the partially opaque fiducial 547. This allows the medical professional to determine that the calibration of the fluoroscopy device 1305 and the position of the third 3D box target 1005-3 is accurate.

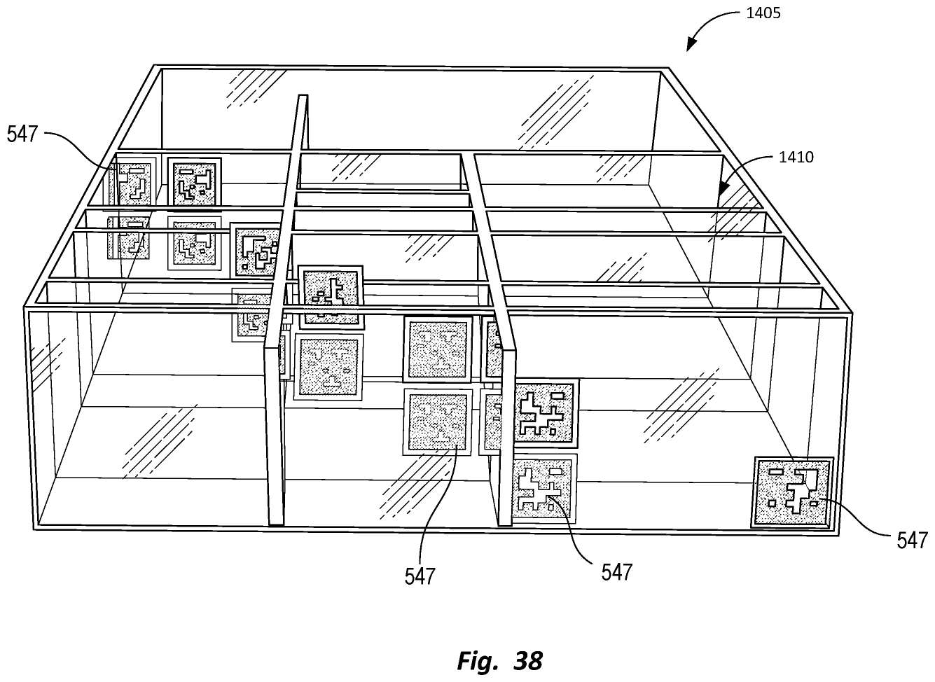

FIG. 38 is a diagram of a partially opaque fiducial tray 1405 including a plurality of partially opaque fiducials 547 formed therein for calculating a pixel-to-meter ratio value on a DICOM file according to an embodiment. As described herein, while performing a procedure, a medical professional will look at a registering computing device 520 or other display device in the computer assisted medical procedure system for guidance. The computer assisted medical procedure system will track whatever tool or medical device the medical professional is using and where it is in relation to the x-ray captured by the fluoroscopy device. In order for that to work, a pixel-to-meter ratio may be determined.

However, when a DICOM file (including an x-ray image of a patient's anatomy) is received at the medical server of the computer assisted surgical system, the x-ray image may not include any information on how physically large the image is, for example, in meters. Some models of fluoroscopy devices include a pixel-to-meter ratio in the DICOM data, but this may not be reliable. Moreover, the nature of the fluoroscopy device 1305 is that the size of the DICOM image (and potentially other image distortions) depends on its proximity to the x-ray emission node and x-ray detection node of the fluoroscopy device 1305. This means that two scans, taken moments apart from each other of the same patient anatomy, at the same angle, but at slightly different distances from the patient will show the anatomy at different sizes. Thus, for each scan and DICOM file, the exact location of the scanline of the fluoroscopy device is to be known in order to know how big to show the image to the medical professional. In an embodiment, there is a pixel-to-meter ratio value for each location of the scanline.

Calculating the pixel-to-meter ratio values can be done by scanning multiple partially opaque fiducials 547 at known distances from each other by the fluoroscopy device 1305. Because the physical size of the partially opaque fiducials 547 as well as the size of each of the partially opaque fiducials 547 in pixels as they come in on the DICOM file, the pixel-to-meter ratio values may be calculated for any location of the scanline.

The partially opaque fiducial tray 1405 allows for this calculation by placing a plurality of partially opaque fiducials 547 placed therein. The partially opaque fiducial tray 1405 may include a plurality of tray wells 1410. Each tray well 1410 may be formed such that a plurality of partially opaque fiducials 547 may be affixed to a back surface of the individual tray wells 1410. Because the back surface of the tray wells 1410 are a predetermined distance from another back surface of another tray well 1410, the partially opaque fiducials 547 are placed in the partially opaque fiducial tray 1405 at a fixed distance know to the medical server. In order to determine the pixel-to-meter ratio values, the medical professional may place this partially opaque fiducial tray 1405 on an operating table and scan the partially opaque fiducials 547 therein with the fluoroscopy device 1305. The resulting x-ray image allows the medical server to calculate the distance between each of the partially opaque fiducials 547 and determine the pixel-to-meter ratio values used during a medical procedure to determine the distance of the patient's anatomy and the pixel-to-meter ratio values of a partially opaque fiducial 547 placed on the patient.

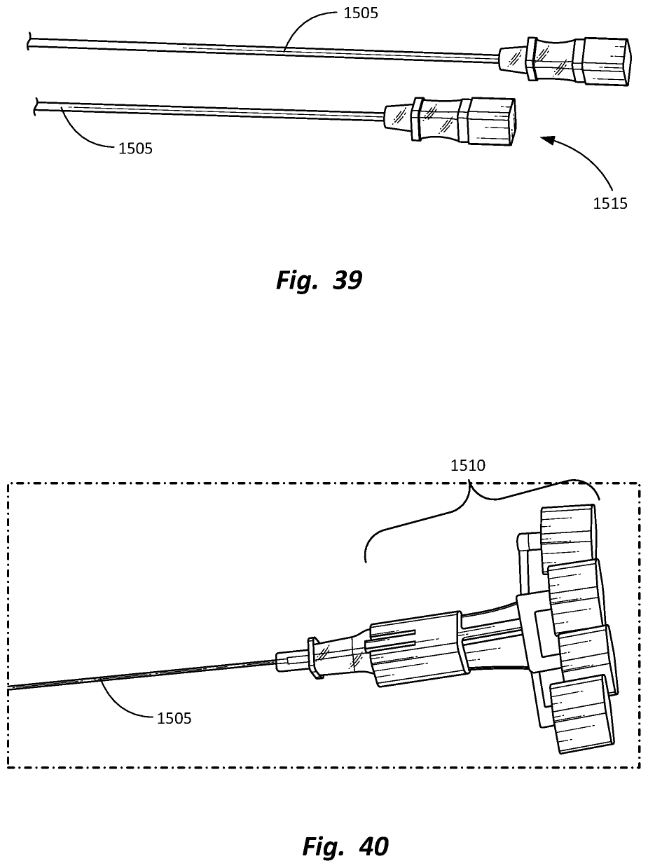

FIG. 39 is a diagram depicting a trackable medical needle 1505 used in a medical procedure according to an embodiment. FIG. 40 is a diagram depicting the trackable medical needle 1505 with a needle tracking array 1510 operatively coupled to a proximal end 1515 of the trackable medical needle 1505 according to an embodiment. In addition to the computer assisted medical procedure system being capable of tracking a fluoroscopy device, the computer assisted medical procedure system may also track other medical devices such as a trackable medical needle 1505 shown in FIGS. 39 and 40. In order to track the exact location of the trackable medical needle 1505, a needle tracking array 1510 may be attached to the proximal end 1515 of the trackable medical needle 1505 that has known shape and geometry. The shape and geometry of the needle tracking array 1510 may be known by medical server of the computer assisted medical procedure system so that it may be detectable within images taken by the primary devices and/or subordinate device. A relative position of the needle tracking array 1510 to the distal end of the trackable medical needle 1505 which can vary depending on the length and type of the trackable medical needle 1505. For example, trackable medical needle 1505 may have different lengths and, in some embodiments, may have a bent distal end. The offsets of the needle tracking array 1510 to the distal end of the trackable medical needle 1505 are calibration values that may be stored in a look-up table stored on a memory device of the medical server as described herein. In embodiment, the type/brand of trackable medical needle 1505 may be used to identify this calibration value defining the distance and orientation of the distal end of the trackable medical needle 1505 to the needle tracking array 1510.

The exact type of trackable medical needle 1505 used for a procedure can be entered by the medical professional manually using a keyboard or mouse at the medical server in an embodiment. In an embodiment, the exact type of trackable medical needle 1505 used for a procedure may be restricted by settings specified for the medical facility such that only certain types and lengths of trackable medical needles 1505 are allowed to be used. Alternatively, supported trackable medical needles 1505 may be modeled for tracking with a primary device and/or subordinate device. Tracking a trackable medical needle 1505 with a long stem, for example, may be used for identifying a model type high-precision tracking while moving is not necessary.

Because the needle tracking array 1510 is fixed to a particular type of trackable medical needle 1505, verifying the calibration may not need to happen often. This calibration process can be done easily by pointing the tip of the trackable medical needle 1505 at a specified point of a fiducial (either an image target or some other type of target such as a fiducial). Since both an image target and needle tracking array 1510 are targets that can be tracked by either of the primary device and subordinate device, pointing the tip of the trackable medical needle 1505 to an exact point on an image target allows the computer assisted surgical system to detect whether the trackable medical needle 1505 is properly calibrated. In a sterile environment, the calibration process may render that exact needle used for calibration unfit for a medical procedure and a replacement trackable medical needle 1505 may be used instead.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure disclosed herein without departing from the spirit and scope of the disclosure.

What is claimed is:

1. A computer assisted medical procedure system, comprising:
at least one registering computing device;
a medical device locator operatively coupled to a medical device placed within a medical procedure theater, wherein the at least one registering computing device determines the location of the medical device locator and medical device relative to a patient; and
a patient position fiducial associated with anatomy of a patient being subjected to a medical procedure;
wherein the at least one registering computing device detects the position of the patient position fiducial relative to the medical device locator in order to determine a relative position of the medical device to the anatomy of the patient.

2. The computer assisted medical procedure system of claim 1, wherein the at least one registering computing device is a head-mounted display device.

3. The computer assisted medical procedure system of claim 1, further comprising:
a medical server configured to maintain a medical image of the patient.

4. The computer assisted medical procedure system of claim 3, wherein the medical server is configured to present the medical image of the patient to a medical professional with an image of the medical device being overlayed on the medical image describing a relative position of the medical device to the anatomy of the patient.

5. The computer assisted medical procedure system of claim 1, wherein the patient position fiducial is partially opaque to fluoroscopy such that the patient position fiducial is represented in an x-ray image captured by a fluoroscopy imaging device.

6. The computer assisted medical procedure system of claim 1, wherein the medical device locator further comprises a quick response (QR) code configured to identify a location where a registering computing device is located within the medical procedure theater.

7. The computer assisted medical procedure system of claim 1, wherein:
the at least one registering computing device comprises a plurality of cameras operatively coupled to a medical server such that the plurality of cameras capture independent images within the medical procedure theater; and
the medical server identifies fixed camera three-dimensional extrinsic points within the medical procedure theater including the medical device locator and triangulates a location of the fixed camera three-dimensional extrinsic points to identify locations of the medical device and the patient position fiducial relative to each other.

8. The computer assisted medical procedure system of claim 7, wherein the plurality of cameras are fixed to a support structure.

9. A computer assisted medical procedure system, comprising:
a medical server;
a plurality of cameras fixed to a support structure placed within a medical procedure theater that each capture independent images within the medical procedure theater, wherein each of the plurality of cameras are operatively coupled to the medical server;
a first medical device locator configured to be operatively coupled to a first medical device placed within the medical procedure theater;
a second medical device locator configured to be operatively coupled to a second medical device positioned within the medical procedure theater; and
a patient position fiducial configured to be placed near anatomy of a patient;
wherein the medical server determines the location of the first medical device and second medical device relative to each other by receiving the captured independent images from the plurality of cameras and identifies fixed camera three-dimensional extrinsic points within the captured independent images;
wherein the medical server detects the position of the patient position fiducial to determine the position of the first medical device and second medical device relative to the anatomy of the patient.

10. The computer assisted medical procedure system of claim 9 further comprising:
the medical server including a medical image database to provide a medical image of the patient during a medical procedure.

11. The computer assisted medical procedure system of claim 10, further comprising:
wherein the medical image of the patient is presented to a medical professional with an image of the second medical device being overlayed on the medical image describing a relative position of the first medical device to the anatomy of the patient.

12. The computer assisted medical procedure system of claim 9, further comprising:
wherein the patient position fiducial is partially opaque to fluoroscopy such that the patient position fiducial is represented in an x-ray image captured by a fluoroscopy imaging device.

13. The computer assisted medical procedure system of claim 9, further comprising:
the first medical device locator including a quick response (QR) code used to identify the first medical device and second medical device and a location of the first medical device and second medical device within the medical procedure theater.

14. The computer assisted medical procedure system of claim 9, wherein the plurality of cameras fixed to the support structure are used to calibrate a fluoroscopy device used to capture an image of the anatomy of the patient for use by medical personnel in performing a medical procedure.

15. A computer assisted medical device calibration system comprising:
a medical server;
a plurality of cameras fixed to a support structure placed within a medical procedure theater that each capture independent images within the medical procedure theater, wherein each of the plurality of cameras are operatively coupled to the medical server; and
a medical device locator operatively coupled to a medical device placed within the medical procedure theater wherein the medical server determines the location of the medical device relative to the plurality of cameras fixed to the support structure by receiving the captured independent images from the plurality of cameras and identifying fixed camera three-dimensional extrinsic points within the captured independent images.

16. The computer assisted medical device calibration system of claim 15 further comprising:
the fixed camera three-dimensional extrinsic points including edges and points on medical device locator operatively coupled to the medical device.

17. The computer assisted medical device calibration system of claim 15 further comprising:
at the medical server, triangulating the identified fixed camera three-dimensional extrinsic points using a first captured image from a first camera of the plurality of cameras and a second captured image from a second camera of the plurality of cameras.

18. The computer assisted medical device calibration system of claim 15 further comprising:
the medical device includes a c-arm type fluoroscopy device with a first medical device locator placed at a x-ray emission node, a second medical device locator placed at an x-ray detection node, and a third medical device locator coupled to a c-arm of the c-arm type fluoroscopy device such that the plurality of cameras fixed to the support structure capture independent images of the first medical device locator, the second medical device locator, and third medical device locator in order to determine a scanline between the x-ray emission node and the x-ray detection node.

19. The computer assisted medical device calibration system of claim 15 further comprising:
the medical device includes a needle and the medical device locator includes a needle location array that has a geometry known by the medical server, wherein the captured independent images from the plurality of cameras fixed to the support structure are used by the medical server to determine the relative position of the needle location array to a tip of the needle.

20. The computer assisted medical device calibration system of claim 19 further comprising:
determining the relative position of the needle location array to a tip of the needle based on needle type input data provide by a medical professional at the medical server.
